(19) United States
(12) Patent Application Publication
(10) Pub. No.: US 2023/0256093 A1
(43) Pub. Date: Aug. 17, 2023

(54) METHOD

(71) Applicant: AUTOLUS LIMITED, London (GB)

(72) Inventors: Marco Della Peruta, London (GB); Matteo Righi, London (GB); Giulia Agliardi, London (GB); Martin Pulé, London (GB); Shaun Cordoba, London (GB); James Sillibourne, London (GB)

(21) Appl. No.: 17/924,345

(22) PCT Filed: May 12, 2021
(86) PCT No.: PCT/GB2021/051134

§ 371 Date: Nov. 9, 2022

(30) Foreign Application Priority Data
May 13, 2020 (GB) 2007044.7

(51) Int. Cl. A61K 39/00; A61K 38/20; A61K 38/19; A61P 35/00

(52) U.S. Cl. CPC A61K 39/4632; A61K 38/208; A61K 39/4631; A61K 39/4611; A61K 38/2046; A61K 38/2086; A61K 38/195; A61P 35/00

(57) ABSTRACT

The present invention provides a method for treating a solid cancer which comprises the step of administering a cell to a subject, wherein the cell comprises a nucleic acid sequence encoding interleukin 12 (IL-12) downstream of a frame-slip motif (FSM) or a translational readthrough motif (TRM).

### CROSS-REFERENCE TO RELATED APPLICATIONS

This is a U.S. National Phase of International Application No. PCT/GB2021/051134, filed May 12, 2021, which claims priority to Great Britain Application No. 2007044.7, filed May 13, 2020.

### FIELD OF THE INVENTION

The present invention relates to chimeric antigen receptor (CAR)-expressing cells, such as CAR-T cells, which secrete interleukin-12 (IL-12). The IL-12-encoding sequence is positioned downstream of a frame-slip motif (FSM) or a translational readthrough motif (TRM) so that IL-12 is secreted at very low levels.

### BACKGROUND TO THE INVENTION

Tumour heterogeneity describes the observation that different tumour cells can show distinct morphological and phenotypic profiles, including cellular morphology, gene expression, metabolism, motility, proliferation, and metastatic potential.

Heterogeneity occurs between patients, between tumours (inter-tumour heterogeneity) and within tumours (intra-tumour heterogeneity). Multiple types of heterogeneity have been observed between tumour cells, stemming from both genetic and non-genetic variability.

Heterogeneity between tumour cells can be further increased due to heterogeneity in the tumour microenvironment. Regional differences in the tumour (e.g. availability of oxygen) impose different selective pressures on tumour cells, leading to a wider spectrum of dominant subclones in different spatial regions of the tumour. The influence of microenvironment on clonal dominance is also a likely reason for the heterogeneity between primary and metastatic tumours seen in many patients, as well as the inter-tumour heterogeneity observed between patients with the same tumour type.

The heterogeneity of cancer cells introduces significant challenges in designing effective treatment strategies.

For example, heterogeneic tumours may exhibit different sensitivities to cytotoxic drugs among different clonal populations. This is attributed to clonal interactions that may inhibit or alter therapeutic efficacy.

Drug administration in heterogeneic tumours will seldom kill all tumour cells. The initial heterogeneic tumour population may bottleneck, such that few drug resistant cells (if any) will survive. This allows resistant tumour populations to replicate and grow a new tumour through the branching evolution mechanism. The resulting repopulated tumour is heterogeneic and resistant to the initial drug therapy used. The repopulated tumour may also return in a more aggressive manner.

There is therefore a need for alternative approaches to address the issue of antigen heterogeneity in solid tumours.

Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

To date, the success of the CAR T cell has largely been in hematological malignancies. A CAR targeted to the B cell antigen CD19 was first used successfully to treat chronic lymphoblastic leukemia (CLL). In August 2017, the FDA approved the use of CART19 (Kymriah) to treat pediatric relapsed or refractory acute lymphoblastic leukemia (ALL) and in October of the same year, another CD19-targeting CAR (Yescarta) was approved by the FDA for adult relapsed or refractory large B cell lymphoma. Additionally, the European Medicines Agency (EMA) also approved the use of both these drugs in June of 2018. However, despite extensive research, CAR T cell therapy for solid tumours has not been nearly as successful.

Overcoming the Hostile Tumour Microenvironment

One of the key hurdles which need to be overcome for a successful cell-based immunotherapeutic treatment of solid cancers is the tumour microenvironment (TME), which has been extensively characterized as hostile for T cells. This is true for CAR-T cells, but also other immunotherapeutic approaches such as using tumour-infiltrating lymphocyctes (TILs) and T-cells expressing engineered T-cell receptors (TCRs).

The glycolytic metabolism of tumour cells renders the environment hypoxic, acidic, low in nutrients and prone to oxidative stress. In an inflammatory environment, tumours cells often upregulate ligands such as programmed cell death ligand 1 (PD-L1) and Galectin-9 that bind to inhibitory receptors on T cells. The tumour microenvironment also relies on stromal cells like cancer associated fibroblasts (CAFs) and suppressive immune cells, including myeloid-derived suppressor cells (MDSCs), tumour associated macrophages (TAMs), tumour associated neutrophils (TANs), mast cells, and regulatory T cells (Tregs). These cells and tumour cells secrete soluble factors like vascular endothelial growth factor (VEGF) and transforming growth factor β (TGFβ), which contribute to abnormal tumour vasculature, promote anti-inflammatory polarization of TAMs and other immune cells, and are implicated in EMT. They also produce reactive oxygen species (ROS) and molecules like lactate, indoleamine 2,3-dioxygenase (IDO), prostaglandin E2 (PGE2), soluble Fas, and adenosine, which contribute to the suppression of the T cell immune response.

Administration of cytokines to polarize the tumour milieu to be more hospitable to T cells and improve CAR T cell recruitment and functionality has been tested in both preclinical and clinical trials. Local delivery of IL-12, which induces inflammatory immune cell recruitment, has been shown to augment the anti-tumor activity of adoptively transferred anti-VEGFR-2 CAR T cells in mice. CAR T cells which constitutively secrete cytokines such as IL-12, termed “armored” CARs have been created to enhance T cell infiltration and function in solid tumors. However, these approaches have limitations: systemic administration of cytokines can be toxic; constitutive production of cytokines may lead to uncontrolled proliferation and transformation (Nagarkatti et al (1994) PNAS 91:7638-7642; Hassuneh et al (1997) Blood 89:610-620).

There is therefore a need for alternative approaches to help immunotherapeutic cells, such as CAR-T cells, survive and persist in the hostile microenvironment of a solid tumour.

### DESCRIPTION OF THE FIGURES

FIG. 1—Schematic diagram showing a classical chimeric antigen receptors (a) Basic schema of a chimeric antigen receptor; (b) First generation receptors; (c) Second generation receptors; (d) Third generation receptors.

FIG. 2: Frame slip motifs and construct design

(A) Diagram illustrating a frame-slip motif where a series of seven uridines are inserted into the transgene sequence, which promotes frame-slip. Frameslipping results in continued transcription/translation of an alternative reading frame, usually in the −1 direction, and the generation of a functional protein. (B) Structure of a construct showing the locations of the transgenes, the frameslip motif (SLIP) and the 2A self-cleaving peptide sequence. (C) Flow cytometric analysis SupT1 cells transduced with constructs containing the RQR8 sort-selection marker followed by either a control sequence (6×U) or a frameslip motif (7×U) and a CD22-CD19 chimeric protein consisting of the ectodomain of CD22 fused to the transmembrane and truncated endodomain of CD19. Introduction of the frameslip motif resulted in dramatically reduced expression of the CD22-CD19 chimera while similar levels of the RQR8 sort selection marker were observed.

FIG. 3: Translational readthrough motifs and construct design.

(A) Examples of known translational readthrough motifs. (B-G) Structure of translational readthrough motif constructs. (B) Translational readthrough construct consisting of two transgenes where the translational readthrough motif is placed 3′ of the first transgene and 5′ of 2A self-cleaving peptide sequence and transgene 2. Expression of transgene 1 will be significantly higher than transgene 2. (B′) Double stop translational readthrough construct where two stop codons are incorporated into the translational readthrough motif to reduce expression levels even further than those achieved with a single stop codon. (C) Universal translational readthrough construct consisting of a translational readthrough motif flanked by two 2A self-cleaving peptide sequences. This construct mitigates sequence-dependent effects that can result in unpredictable levels of translational readthrough. (D) Combinatorial approach where a translational readthrough motif is combined with an attenuated signal peptide sequence to reduce the level of expression of transgene 2 even further. (E and E′) Compound translational readthrough motifs where multiple motifs are placed in series to produce a cascade of reduced transgene expression. (E) Multiple transgenes are expressed from a single cassette using translational readthrough motifs and 2A self-cleaving peptide sequences. (E′) Self-cleaving peptide sequences are used to separate the translational readthrough motifs, which are 5′ of transgene 2.

FIG. 4: Schematic diagram illustrating the drastic reduction in IL-12 secretion in cells transduced with a vector in which the IL-12-encoding sequence is placed downstream of a “stop-skip” sequence (SS), i.e. a frame-slip motif or a translational readthrough motif in combination with a stop codon.

FIG. 5: ELISA showing concentration of IL-12 in the supernatant of cells either left untransduced (NT) or transduced with: a construct co-expressing a CAR and IL-12 (CAR-2A-IL12); or a construct co-expressing a CAR and IL-12 in which the IL-12 encoding sequence is placed downstream of a “stop-skip” sequence (CAR-SS-IL12).

FIG. 6: Graph showing the weight of mice following administration of cells either left untransduced (NT) or transduced with: a construct co-expressing a CAR and IL-12 (CAR-2A-IL12); or a construct co-expressing a CAR and IL-12 in which the IL-12 encoding sequence is placed downstream of a “stop-skip” sequence (CAR-SS-IL12).

FIG. 7: Graphs showing tumour size over time in mice following administration of cells either left untransduced (NT) or transduced with: a construct expressing a GD2 CAR alone (CAR); a construct co-expressing a GD2 CAR and IL-12 (CAR-2A); a construct co-expressing a GD2 CAR and IL-12 in which the IL-12 encoding sequence is placed downstream of a “stop-skip” sequence (CAR-SS); or a construct co-expressing a CAR to an irrelevant target antigen and IL-12 in which the IL-12 encoding sequence is placed downstream of a “stop-skip” sequence (MR1-SS).

### SUMMARY OF ASPECTS OF THE INVENTION

The present inventors have shown in a mouse model that administration of engineered cells expressing high-level IL-12 is toxic. They have also shown that it is possible to drastically reduce the level of expression of IL-12 by the inclusion of a motif upstream of the IL-12-encoding sequence. These ultra-low IL-12 expressing cells do not cause significant toxicity in the mouse model. Moreover, in a solid tumour model, ultra-low IL-12 expressing CAR-T cells exhibit more potent anti-tumour activity that cells expressing CAR alone.

Thus, in a first aspect, the present invention provides a method for treating a solid cancer which comprises the step of administering a cell to a subject, wherein the cell comprises a nucleic acid sequence encoding interleukin 12 (IL-12) downstream of a frame-slip motif (FSM) or a translational readthrough motif (TRM).

The method may comprise the following steps:
(i) isolation of a cell sample from a subject;
(ii) transfection or transduction of cells from the cell sample with a nucleic acid sequence encoding interleukin 12 downstream of a frame-slip motif (FSM) or a translational readthrough motif (TRM); and
(iii) administering transduced or transfected cells from step (ii) to the subject.

The nucleic acid sequence encoding IL-12, may encode “flexi-IL-12”: a fusion between IL-12α and IL-12β subunits, joined by a linker. Flexi-IL-12 may comprise the sequence shown as SEQ ID No. 1.

Where the nucleic acid sequence encoding interleukin 12 (IL-12) is downstream of a frame-slip motif (FSM), the FSM may comprise a repeat of uracil, thymine or guanine bases. For example, the FSM may comprise the sequence UUUUUUU (SEQ ID No. 2).

The FSM may also comprise a stop codon. For example, the FSM may comprise one of the following sequences:

(SEQ ID NO. 3)
UUUUUUUGA
(SEQ ID NO. 4)
UUUUUUUAG
(SEQ ID NO. 5)
UUUUUUUAA.

Where the nucleic acid sequence encoding interleukin 12 (IL-12) is downstream of a translational readthrough motif (TRM), the TRM may comprise the sequence STOP-CUAG or STOP-CAAUUA, in which “STOP” is a stop codon.

The translational readthrough motif may comprise one of the following sequences:

(SEQ ID No. 6)
UGA-CUAG
(SEQ ID No. 7)
UAG-CUAG
(SEQ ID No. 8)
UAA-CUAG
(SEQ ID No. 9)
UGA-CAAUUA
(SEQ ID No. 10)
UAG-CAAUUA
(SEQ ID NO. 11)
UAA-CAAUUA

The cell may be a tumour infiltrating immune cell, such as a T-cell, natural killer (NK) cell, NKT cell, cytokine-induced killer (CIK) cell, monocyte, macrophage or tumour-infiltrating lymphocyte (TIL).

The cell may express a chimeric antigen receptor (CAR) or engineered T-cell receptor (TCR), for example, a CAR or engineered TCR which binds to one of the following target antigens: disialoganglioside (GD2); epidermal growth factor receptor (EGFR), Epithelial cell adhesion molecule (EpCAM), Glypican 3 (GPC3), human epidermal growth factor receptor (HER2), L1CAM, Mucin 1 (MUC1), Prostate-specific membrane antigen (PSMA).

In one embodiment of the invention the cell expresses a CAR which binds to GD2 and has an antigen-binding domain which comprises

a) a heavy chain variable region (VH) having complementarity determining regions (CDRs) with the following sequences:

CDR1-
(SEQ ID No. 12)
YNIH;
CDR2-
(SEQ ID No. 13)
VIWAGGSTNYNSALMS
CDR3-
(SEQ ID No. 14)
RSDDYSWFAY;

and

b) a light chain variable region (VL) having CDRs with the following sequences:

CDR1-
(SEQ ID No. 15)
RASSSVSSSYLH;
CDR2-
(SEQ ID No. 16)
STSNLAS
CDR3-
(SEQ ID NO. 17)
QQYSGYPIT.

In another embodiment of the invention, the cell expresses a CAR which binds PSMA and has an antigen-binding domain which comprises

a) a heavy chain variable region (VH) having complementarity determining regions (CDRs) with the following sequences:

| | CDR1 |
|---|---|
| (SEQ ID No. 18) | |
| | SSWMN; |
| | CDR2 |
| (SEQ ID No. 19) | |
| | RIYPGDGDTNYAQKFQG |
| | CDR3 |
| (SEQ ID No. 20) | |
| | GTGYLWYFDV; |

and

b) a light chain variable region (VL) having CDRs with the following sequences:

| | CDR1 |
|---|---|
| (SEQ ID No. 21) | |
| | RASQDINENLA; |
| | CDR2 |
| (SEQ ID No. 22) | |
| | YTSNRAT |
| | CDR3 |
| (SEQ ID NO. 23) | |
| | QQYDNLPFT. |

The cell may also express:
a dominant negative SHP2; and/or a dominant negative TGFβ receptor;
a chimeric cytokine receptor; and/or
one or more additional cytokine(s) or chemokine(s).

The cell may express one or more of the following: IL-7, IL-15, CCL19, CXCL12. In particular, the cell may express IL-7 and CCL19; or IL-15 and/or CXCL12.

The nucleic acid encoding the or each additional cytokine(s) or chemokine(s) may be positioned upstream of the frame-slip motif (FSM) or translational readthrough motif (TRM).

Alternatively, the nucleic acid encoding the or each additional cytokine(s) or chemokine(s) may be positioned downstream of the frame-slip motif (FSM) or translational readthrough motif (TRM).

The method of the invention may be for the treatment of a solid cancer such as: small cell lung cancer (SCLC), melanoma, renal cell cancer (RCC), hepatocellular carcinoma (HCC), ovarian cancer, pancreatic cancer, neuroblastoma or osteosarcoma.

The CAR or engineered TCR may bind a target antigen which has heterogeneous expression on the solid tumour.

In a second aspect, the present invention provides a method for inducing epitope spreading in an anti-tumour immune response in a subject, which comprises the step of administering a plurality of cells as defined in the first aspect of the invention to the subject.

In a third aspect, the present invention provides a method for inducing infiltration of immune cells into a tumour mass in a subject which comprises the step of administering a plurality of cells as defined in the first aspect of the invention to the subject, such that they induce an anti-tumour immune response.

In a fourth aspect, there is provided a cell for use in treating a solid cancer, the cell comprising a nucleic acid sequence encoding interleukin 12 (IL-12) downstream of a frame-slip motif (FSM) or a translational readthrough motif (TRM).

In a fifth aspect, there is provided the use of a cell in the manufacture of a medicament for treating a solid cancer, wherein the cell comprises a nucleic acid sequence encoding interleukin 12 (IL-12) downstream of a frame-slip motif (FSM) or a translational readthrough motif (TRM).

In a sixth aspect, there is provided a cell which expresses a chimeric antigen receptor (CAR) and comprises; a nucleic acid sequence encoding interleukin 12 (IL-12) downstream of a frame-slip motif (FSM) or a translational readthrough motif (TRM); and one or more heterologous nucleic acid sequence(s) encoding one or more additional cytokine(s) or chemokine(s).

The one or more heterologous nucleic acid sequence(s) may encode one or more of the following: IL-7, IL-15, CCL19, CXCL12.

For example, the one or more heterologous nucleic acid sequence(s) may encode IL-7 and CCL19.

Alternatively, the one or more heterologous nucleic acid sequence(s) may encode IL- and/or CXCL12.

The nucleic acid sequence encoding an additional cytokine or chemokine may be positioned upstream or downstream of the frame-slip motif (FSM) or translational readthrough motif (TRM). Where there are two or more nucleic acid sequences encoding an additional cytokine(s) or chemokine(s), one or more may be upstream of the FSM or TRM, so it is/they are expressed at a high level; and one or more may be downstream of the FSM or TRM, so it is/they are expressed at a low level.

In a seventh aspect, there is provided a nucleic acid construct comprising:
(i) a nucleic acid sequence encoding a chimeric antigen receptor (CAR); and
(ii) a nucleic acid sequence encoding interleukin 12 (IL-12) downstream of a frame-slip motif (FSM) or a translational readthrough motif (TRM);
(vi) one or more nucleic acid sequence(s) encoding one or more additional cytokine(s) or chemokine(s) as defined above.

In an eighth aspect, there is provided a vector comprising a nucleic acid construct according to the seventh aspect of the invention.

In a ninth aspect, there is provided a kit of vectors, each encoding one or more nucleic acid sequence(s) as defined above.

In a tenth aspect. there is provided a method for making a cell according to the sixth aspect of the invention, which comprises the step of transfecting or transducing a cell with a nucleic acid construct according to the seventh aspect, a vector according to the eighth aspect or a kit of vectors according to the ninth aspect, ex vivo.

The present invention provides cells for the treatment of cancers, in particular solid tumours, which secrete IL-12 at very low levels.

Unlike previously developed “armoured” CAR-T cells the level of secretion of IL-12 is reduced by the incorporation of a frame-slip motif or a translational readthrough motif upstream of the IL-12 encoding sequence. The present inventors have shown that the local expression of low-level IL-12 greatly enhances the anti-tumour response but does not cause problematic levels of toxicity.

The local secretion of IL-12 has two effects: firstly, it increases the resistance of the CAR-T cells to the hostile tumour environment, increasing the proliferation and persistence of CAR T cells; secondly, it has an adjuvant effect, causing a general activation of the anti-tumour immune response.

In this respect, the release of IL-12 has various effects during an immune response, including 1) enhancing proliferation of T cells and NK cells, 2) increasing cytolytic activities of T cells, NK cells, and macrophages, 3) activating T helper 1 (Th1) cells, and 4) inducing production of IFN-γ and other cytokines. In addition to activating CAR-T cells, the it is thought that the simultaneous local expression of low-level IL-12 by CAR-T cells will lead to “epitope spreading” i.e. the priming of immune responses against additional target antigens present on tumour cells. The secretion of IL-12 by CAR-T cells may also induce the infiltration of host immune cells into the tumour mass. The anti-tumour immune response is therefore activated through a combination of direct cell killing by CAR-T cells, infiltration of immune cells into the tumour and the induction of an anti-cancer immune response from existing host immune cells against other tumour-associated antigens.

The induction of epitope spreading is particularly useful for the treatment of solid tumours, which often show heterogeneity in expression of target antigen.

Further Aspects

The present invention also provides additional aspects which are summarised in the following numbered paragraphs:

1. A method for treating small cell lung cancer, which comprises the step of administering a cell to a subject, wherein the cell expresses a GD2 CAR, dominant negative SHP2 (dnSHP2) and dominant negative TGFβRII.

2. A method according to paragraph 1, wherein the cell also expresses a chimeric cytokine receptor (CCR).

3. A method according to paragraph 2, where in the CCR has IL-7R endodomains.

4. A method according to paragraph 1, wherein the cell comprises a first nucleic acid construct having the following general structure:

CAR-coexpr1-dnSHP-coexpr2-dnTGFβR,

dnSHP-coexpr1-CAR-coexpr2-dnTGFβR,

CAR-coexpr1-SG-coexpr2-dnSHP-coexpr3-dnTGFβR,

dnSHP-coexpr1-SG-coexpr2-CAR-coexpr2-dnTGFβR,

in which:

dnSHP is a nucleic acid sequence encoding dominant negative SHP-2

“coexpr1”, “coexpr2” and “coexpr3” which may be the same or different, are nucleic

acid sequences enabling coexpression of each polypeptide as separate entities

“dnTGFβR” is a nucleic acid sequence encoding a dominant negative TGFβ receptor;

“CAR” is a nucleic acid sequence encoding an anti-GD2 chimeric antigen receptor; and

“SG” is a nucleic acid sequence encoding a suicide gene such as RQR8.

5. A method according to paragraph 2 or 3, wherein the cell comprises a second nucleic acid construct having the following general structure:

CAR-coexpr1-CCR, or

CAR-coexpr1-SG-coexpr2-CCR

in which:

“CAR” is a nucleic acid sequence encoding an anti-GD2 chimeric antigen receptor;

“coexpr1” and “coexpr2” and “coexpr3” which may be the same or different, are nucleic acid sequences enabling co-expression of each polypeptide as separate entities;

“CCR” is a nucleic acid sequence encoding a chimeric cytokine receptor; and

“SG” is a nucleic acid sequence encoding a suicide gene such as RQR8.

6. A method according to paragraph 1 which comprises the following steps:
(i) isolation of a cell-containing sample from the subject;
(ii) transduction or transfection of the cells with a vector expressing a first nucleic acid construct as defined in paragraph 4; and
(iii) administration of cells from step (ii) to the subject.

7. A method according to paragraph 2 or 3 which comprises the following steps:
(i) isolation of a cell-containing sample from the subject;
(ii) transduction or transfection of the cells with a first vector expressing a first nucleic acid construct as defined in paragraph 4 and a second vector expressing a second nucleic acid construct as defined in paragraph 5; and
(iii) administration of cells from step (ii) to the subject.

8. A cell for use in treating small cell lung cancer (SCLC), wherein the cell expresses a GD2 CAR, dominant negative SHP2 (dnSHP2) and dominant negative TGFβRII.

5 9. The use of a cell in the manufacture of a medicament for treating small cell lung cancer (SCLC), wherein the cell expresses a GD2 CAR, dominant negative SHP2 (dnSHP2) and dominant negative TGFβRII.

The following detailed description, as it relates to nucleic acid and polypeptide sequences, polypeptide components, vectors, cells methods etc applies equally to the aspects laid out in the above paragraphs as to the aspects of the invention in the claims.

### DETAILED DESCRIPTION

The present invention provides a cell which expresses interleukin 12 (IL-12) at very low levels. The cell comprises a nucleic acid encoding IL-12 downstream of a frame-slip motif (FSM) or a translational readthrough motif (TRM).

IL-12 and Other Cytokines/Chemokines

The present invention provides a cell which comprises a nucleic acid sequence encoding interleukin 12 (IL-12) downstream of a frame-slip motif (FSM) or a translational readthrough motif (TRM). The cell may also comprise a heterologous nucleic acid sequence encoding a cytokine other than IL-12 and/or a chemokine.

Interleukin 12 (IL-12) is an interleukin that is naturally produced by dendritic cells, macrophages, neutrophils, and human B-lymphoblastoid cells in response to antigenic stimulation. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ.

IL-12 plays an important role in the activities of natural killer cells and T lymphocytes. IL-12 mediates enhancement of the cytotoxic activity of NK cells and CD8+ cytotoxic T lymphocytes.

IL-12 is a potent immunomodulatory cytokine of particular interest for modulating the tumour microenvironment redirecting the immune response against cancer. IL-12 is systemically toxic therefore methods for producing IL-12 locally are of interest.

IL-12 is a heterodimeric cytokine encoded by two separate genes, IL-12A (p35) and IL-12B (p40). The active heterodimer (referred to as ‘p70’), is formed following protein synthesis.

The cell of the present invention may comprise a heterologous nucleic acid sequence encoding IL-12A and/or IL-12B. The sequence for human IL-12A is available from Uniprot Accession number P29459. A portion of this sequence, lacking the signal peptide, is shown below as SEQ ID No. 24. The sequence for human IL-12B is available from Uniprot Accession number P29460. A portion of this sequence, lacking the signal peptide, is shown below as SEQ ID No. 25.

(human IL-12A)
SEQ ID No. 24
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNAS
(human IL-12B)
SEQ ID No. 25
WELKKDVYVVELDWYPDAPGEMVVLTCDTPEEDGITWTLDOSSEVLGSGK
TLTIQVKEFGDAGQYTCHKGGEVLSHSLLLLHKKEDGIWSTDILKDQKEP
KNKTFLRCEAKNYSGRFTCWWLTTISTDLTFSVKSSRGSSDPQGVTCGAA
TLSAERVRGDNKEYEYSVECQEDSACPAAEESLPIEVMVDAVHKLKYENY
TSSFFIRDIIKPDPPKNLQLKPLKNSRQVEVSWEYPDTWSTPHSYFSLTF
CVQVQGKSKREKKDRVFTDKTSATVICRKNASISVRAQDRYYSSSWSEWA
SVPC

The heterologous nucleic acid sequence may encode “flexi-IL-12”, which is a fusion between the human IL-12α (p35) and IL-12β (p40) subunits, joined by a linker. A suitable flexi-IL-12 sequence is shown below as SEQ ID No. 1.

(a flexi-IL-12 sequence)
SEQ ID No. 1
METDTLLLWVLLLWVPGSTGMWIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCSGGGGSGGGGSGGGGSRNLPLAT
PDPGMFPCLHHSQNLLRAVSNMLQKARQTLEFYPCTSEEIDHEDITKDKT
STVEACLPLELTKNESCLNSRETSFITNGSCLASRKTSFMMALCLSSIYE
DSKMYQVEFKTMNAKLLMDPKRQIFLDQNMLAVIDELMQALNFNSETVPQ
KSSLEEPDFYKTKIKLCILLHAFRIRAVTIDRVMSYLNAS

In SEQ ID No. 1, the signal peptide, which is the signal peptide from Murine kappa chain V-III region MOPC 63 (Uniprot P01661), is shown in bold; and the serine-glycine linker is in bold and underlined. Flexi-IL12 may comprise the IL-12A and B sequences shown in SEQ ID No. 1 but with a different signal peptide and/or linker sequence. It may have the general structure:

SP-IL12A-L-IL12B

where SP is a signal peptide, IL12A is human IL-12A; Lisa linker sequence, such as a glycine-serine linker; and IL12B is human IL-12B.

The heterologous nucleic acid sequence may encode one of the sequences shown as SEQ ID No. 1, 24 or 25 or a variant thereof. The variant sequence may have at least 80, 85, 90, 95, 98 or 99% sequence identity to SEQ ID No. 1, 24 or 25, provided that the variant sequence retains IL-12 function when expressed in vivo. For example, the variant sequence may retain the capacity to enhance the activity of cytotoxic T cells in vivo and/or the variant sequence may stimulate the production of interferon-gamma (IFN-γ) by T cells.

The cell may comprise one or more heterologous nucleic acid(s) encoding one or more cytokines in addition to IL-12. The nucleic acid sequence(s) may, for example, encode a cytokine which enhances the inflammatory response and/or increases the efficacy of CAR-T cell therapy. The cytokine may be selected from the following: IL-7, IL15, IL-17A, IL-18, IL-2, GM-CSF and IL-21. In particular, the additional cytokine may be IL-7.

IL-7

IL-7 is a cytokine important for B and T cell development. IL-7 stimulates the differentiation of multipotent (pluripotent) hematopoietic stem cells into lymphoid progenitor cells and stimulates proliferation of all cells in the lymphoid lineage (B cells, T cells and NK cells.

11-7 and the hepatocyte growth factor (HGF) form a heterodimer that functions as a pre-pro-B cell growth-stimulating factor. This cytokine is found to be a cofactor for V(D)J rearrangement of the T cell receptor beta (TCRß) during early T cell development. The amino acid sequence of human IL-7 is available from UniProt (Accession No. P13232) and shown below as SEQ ID No. 26.

(human IL-7)
SEQ ID No. 26
MFHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEH

IL-15

Interleukin-15 (IL-15) is a cytokine with structural similarity to Interleukin-2 (IL-2). Like IL-2, IL-15 binds to and signals through a complex composed of IL-2/IL-15 receptor beta chain and the common gamma chain. IL-15 is secreted by mononuclear phagocytes and other cells following infection by virus(es) and regulates the activation and proliferation of T and natural killer (NK) cells.

The amino acid sequence of human IL-15 is available from UniProt (Accession No. P40933) and shown below as SEQ ID No. 27.

(human IL-15)
SEQ ID No. 27
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

The amino acid sequences of IL-17A, IL-18, IL-2, Granulocyte-macrophage colony-stimulating factor (GM-CSF) and IL-21 are available from UniProt, as shown in the table below.

| | UniProt Accession |
|---|---|
| Cytokine | No. |
| IL-17A | Q16552 |
| IL-18 | Q14116 |
| IL-2 | P60568 |
| GM-CSF | P04141 |
| IL-21 | Q9HBE4 |

The one or more heterologous nucleic acid sequence(s) may encode one or more chemokines.

Chemokines are a family of small cytokines secreted by cells. Their name is derived from their ability to induce directed chemotaxis in nearby responsive cells; they are chemotactic cytokines. Chemokines released by infected or damaged cells form a concentration gradient. Attracted cells move through the gradient towards the higher concentration of chemokine.

Chemokines are all approximately 8-10 kilodaltons in mass and have four cysteine residues in conserved locations that are key to forming their 3-dimensional shape. Some chemokines are considered pro-inflammatory and can be induced during an immune response to recruit cells of the immune system to a site of infection. Examples are: CXCL-8, CCL2, CCL3, CCL4, CCL5, CCL11, CXCL10. Other chemokines are considered homeostatic and are involved in controlling the migration of cells during normal processes of tissue maintenance or development. These include: CCL14, CCL19, CCL20, CCL21, CCL25, CCL27, CXCL12 and CXCL13. This classification is not strict; for example, CCL20 can act also as pro-inflammatory chemokine.

Chemokines have been classified into four main subfamilies: CXC, CC, CX3C and XC. All of these proteins exert their biological effects by interacting with G protein-linked transmembrane receptors called chemokine receptors, that are selectively found on the surfaces of their target cells

The cell may express a homeostatic chemokine such as CCL19 or CXCL12.

Chemokine (C-C motif) ligand 19 (CCL19) is a small cytokine belonging to the CC chemokine family that is also known as EB11 ligand chemokine (ELC) and macrophage inflammatory protein-3-beta (MIP-3-beta). CCL19 elicits its effects on its target cells by binding to the chemokine receptor chemokine receptor CCR7. It attracts certain cells of the immune system, including dendritic cells and antigen-engaged B cells and CCR7+ central-memory T-cells. The amino acid sequence for human CCL19 is available from UniProt (Accession number Q99731) and shown below as SEQ ID No. 28.

(CCL19)
SEQ ID No. 28
MALLLALSLLVLWTSPAPTLSGTNDAEDCCLSVTQKPIPGYIVRNFHYLL
IKDGCRVPAVVFTTLRGRQLCAPPDQPWVERIIQRLQRTSAKMKRRSS

C-X-C motif chemokine 12 (CXCL12), stromal cell-derived factor 1 (SDF1), is produced in two forms, CXCL12a and CXCL12b, by alternate splicing of the same gene. Chemokines are characterized by the presence of four conserved cysteines, which form two disulfide bonds. The CXCL12 proteins belong to the group of CXC chemokines, whose initial pair of cysteines are separated by one intervening amino acid. In addition, the first 8 residues of the CXCL12 N-terminal serve as a receptor binding site, and the RFFESH motif (residues 12-17) in the loop region function as a docking site for CXCL12 receptor binding.

CXCL12 is expressed in many tissues in mice including brain, thymus, heart, lung, liver, kidney, spleen, and bone marrow. CXCL12 is strongly chemotactic for lymphocytes. The amino acid sequence for human CXCL12 is available from UniProt (Accession number P48061) and shown below as SEQ ID No. 29.

(CXCL12)
SEQ ID No. 29
MNAKVVVVLVLVLTALCLSDGKPVSLSYRCPCRFFESHVARANVKHLKIL
NTPNCALQIVARLKNNNRQVCIDPKLKWIQEYLEKALNKRFKM

Stop Skip Motifs

Frame-Slip

During transcription, RNA polymerase catalyzes incorporation of nucleotides into growing RNA chains on the basis of complementarity to the DNA template. When RNA polymerase encounters a stretch of repeated bases, however, slippage or ‘stuttering’ can occur. Transcription slippage is utilized in nature, for example for regulation of the Escherichia coli pyrBI and codBA operons, expression of the P gene in paramyxoviruses and decoding of the cellular dnaX gene of Thermus thermophiles.

When RNA polymerase slips this can result in the synthesis of an mRNA encoding an alternative reading frame because it lacks one (or maybe two) of the repeated bases.

The nucleic acid construct of the present invention may comprise a transcriptional frameslip site, such that transcription of the downstream transgene only occurs if these is slippage of the RNA polymerase resulting in mRNA encoding an alternative reading frame.

Translation of an mRNA sequence to protein is a complex process involving the orchestration of ribosomes, initiation and elongation factors, aminoacyl transfer RNAs (aa-tRNAs), aminoacyl tRNA synthetases and release factor. The initiation of translation begins when a complex of factors binds to the 5′ end of the mRNA, which then results in the recruitment of 40S ribosomes and the scanning of the mRNA. When the complex of initiation factors and 40S ribosome encounters a codon (a triplet of nucleotides) encoding the amino acid methionine (AUG codon), the 60S ribosome is recruited and polypeptide synthesis begins with the pairing of cognate tRNAs loaded with the appropriate amino acid. Initiation of translation can occur at alternative start codons other than AUG, but it is the most frequently used start codon. Extension of the polypeptide occurs in a cyclical manner, whereby a tRNA binds to its cognate codon, a peptide bond forms between the newly added amino acid and the extending polypeptide, and the polypeptide translocates to expose the next codon.

During translation, ribosome pausing may occur where the ribosome arrests at a particular codon. Ribosome pausing can promote mRNA degradation by a nucleocytic pathway or it can induce a slip in the −1 or −2 direction. Repetitive sequences within an mRNA, such as UUUUUUA, are known to induce translational frameslipping, and this sequence is present at the 3′ end of the group-specific antigen gene (gag) and polymerase (pol) gene of the human immunodeficiency virus (HIV). Translational frameslipping −1 of the HIV gag/pol gene results in the expression of the Gag-Pol polyprotein.

The nucleic acid construct of the present invention may comprise a translational frameslipping site, such that translation of the downstream transcript only occurs if these is a frameslip by the ribosome.

The frame-slip site may comprise a stretch of bases of the same type.

The frame-slip motif may be placed upstream and/or downstream of a cleavage site in the nucleic acid construct. The frame-slip motif is located between first and second transgenes in the nucleic acid construct.

The frame-slip motif may be used alone. In this embodiment, the second transgene may be placed out of frame downstream of the frame-slipping site, such that frame-slip is needed for transcription or translation of the second transgene.

The motif may, for example comprise a stretch of 5, 7, 8, 10 or 11 bases. The site may, for example, comprise a repeat of uracil, adenosine or guanine bases. The site may, for example, comprise the sequence shown as SEQ ID No. 2

| (SEQ ID NO. 2) | |
|---|---|
| | UUUUUUU. |

Alternatively, the frame-slip site may be used in combination with a stop codon. In this embodiment, a stop codon is positioned in frame downstream of the frame-slip site, such that frame-slipping is required for the stop codon to be ignored.

The stop codon may be UGA, UAG or UAA. The frame-slip site may comprise a repeat of uracil, adenosine or guanine bases in multiples of three, for example, 3, 6 or 9 repeats of uracil, adenosine or guanine bases.

Examples of frame-slip site/stop codon combinations are shown as SEQ ID No. 3, 4 and 5.

| (SEQ ID NO. 3) | |
|---|---|
| | UUUUUUUGA |
| (SEQ ID NO. 4) | |
| | UUUUUUUAG |
| (SEQ ID NO. 5) | |
| | UUUUUUUAA. |

Translational Readthrough

Translation terminates when the ribosome encounters a UGA, UAG or UAA stop codon. At this point, release factor recognises the stop codon and facilitates dissociation and recycling of the ribosome. Termination of translation usually occurs with high fidelity, with recoding of the stop codon, due to competition between release factor and a near cognate tRNA, and continued extension of the polypeptide only occurring 0.1% of the time. However, an elevated level of stop codon of recoding, which results in translational readthrough, has been reported in certain genes. In some cases, this has resulted in the generation of a longer polypeptide with additional functional motifs, in a process referred to as functional readthrough.

Translational readthrough occurs when release factor 1 (RF1) fails to recognise a stop codon and a near cognate aa-tRNA inserts an amino acid into the extending polypeptide, thereby suppressing the stop codon. The local concentration of release factor and the aa-tRNAs affects the level of stop codon suppression and translational readthrough, with low concentrations of release factor promoting translational readthrough. In mammals suppression of the UAG stop codon results in the insertion of a tryptophan, arginine or cysteine residue.

One of the earliest discovered examples of stop codon suppression is the rabbit beta globin gene, where translational readthrough results in the addition of 22 amino acids to the C-terminus of the protein 3.

The frequency of translational readthrough depends on a number of factors including: 1) the stop codon used (UGA, UAG or UAA); 2) the immediate sequence flanking the stop codon, with the six nucleotides upstream and downstream of the stop codon being particularly important and; 3) the presence of cis-acting sequences in the 3′ end of the mRNA.

The termination efficiency of the three stop codons varies, with UAA being the strongest stop codon and UGA being the weakest and hierarchy of termination efficiency is defined as UAA>UAG>UGA. Consequently, the highest level of translational readthrough is exhibited with the UGA stop codon and the lowest with the UAA stop codon.

Sequence analysis of genes exhibiting translational readthrough has identified at least two different motifs that promote stop codon suppression and sustained translation: STOP-CUAG and STOP-CAAUUA (where stop can be UGA, UAG or UAA). The level of translational readthrough is dependent on the stop codon used: UGA supports the highest level of translational readthrough and decreasing levels of readthrough are obtained from the UAG and UAA stop codons, so the overall hierarchy of readthrough is UGA>UAG>UAA.

The nucleic acid construct of the present invention may comprise one of the sequences shown as SEQ ID No. 6 to 11.

| (SEQ ID No. 6) | |
|---|---|
| | UGACUAG |
| (SEQ ID No. 7) | |
| | UAGCUAG |
| (SEQ ID No. 8) | |
| | UAACUAG |
| (SEQ ID No. 9) | |
| | UGACAAUUA |
| (SEQ ID No. 10) | |
| | UAGCAAUUA |
| (SEQ ID NO. 11) | |
| | UAACAAUUA |

The translational readthrough site may be located between first and second transgenes in the nucleic acid construct. The translational readthrough site may be placed upstream and/or downstream of a cleavage site in the nucleic acid construct. The translational readthrough site may be flanked by cleavage sites (FIG. 3C). Two or more translational readthrough sites may be used, for example, either positioned next to each other (FIG. 3B′), or flanking a cleavage site (FIG. E′).

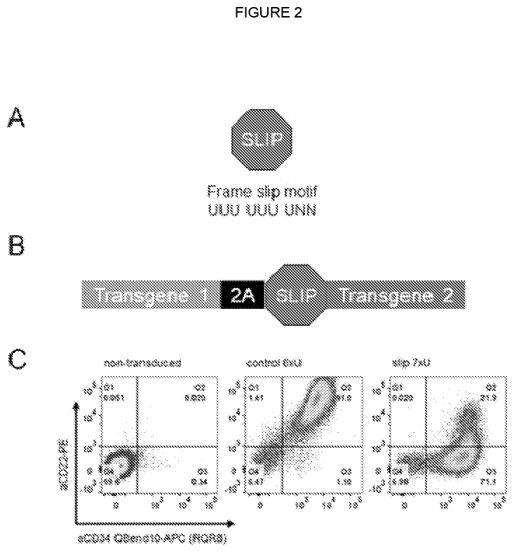

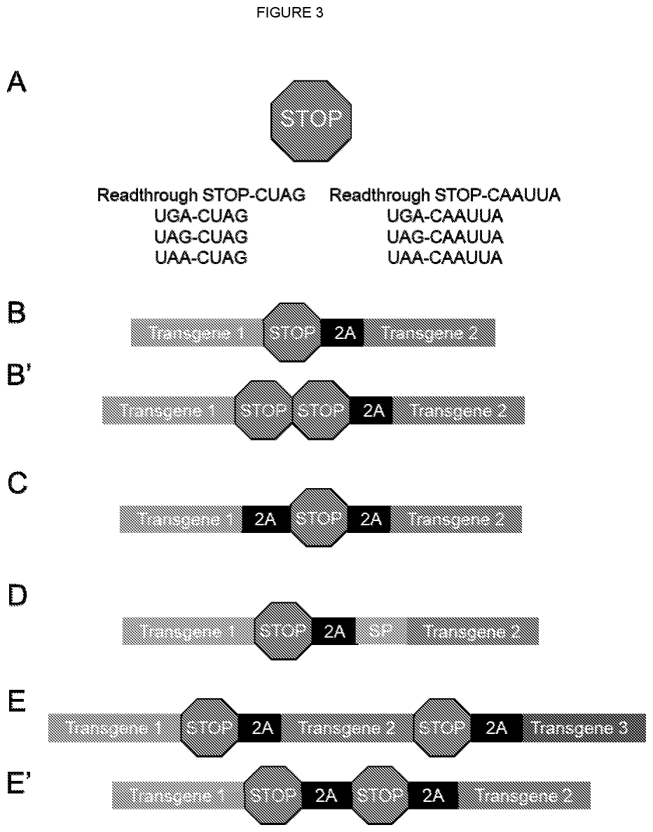

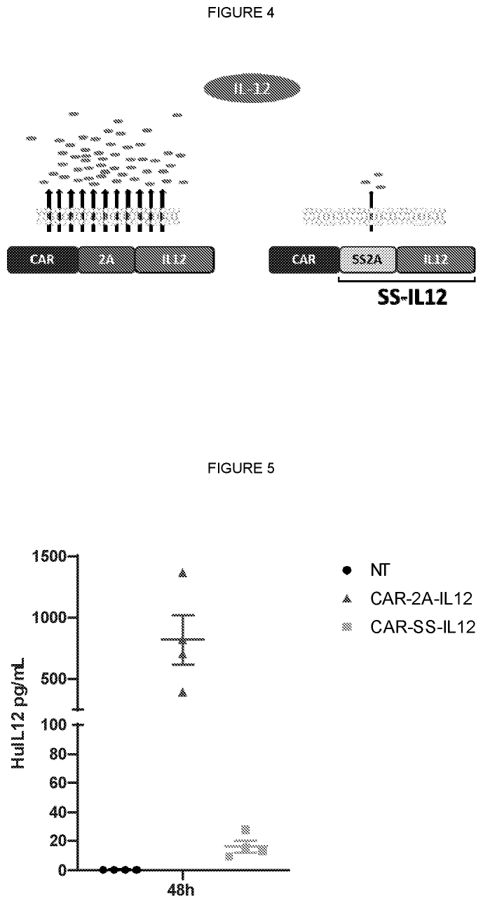

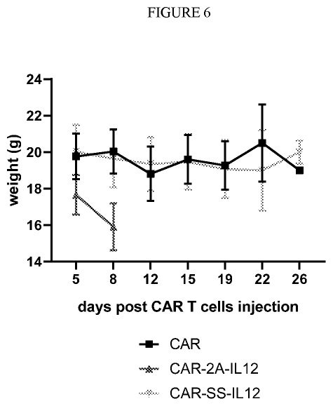

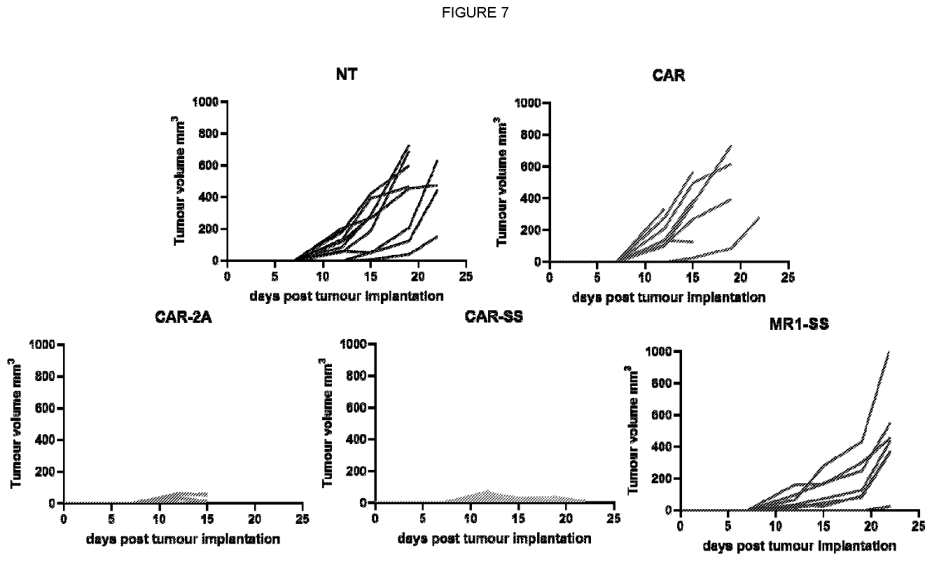

To reduce the expression level of a downstream transgene further, multiple stop codons can inserted 5′ of the translational readthrough motif (FIG. 2B). An example multiple stop translational readthrough motif is shown as SEQ ID NO. 30, where two UGA stop codons are positioned 5′ of the CUAG readthrough motif.

| (SEQ ID NO. 30) | |
|---|---|
| | UGAUGACUAG |

The above sequences are presented as RNA, but in the construct they may be the equivalent DNA sequence which produces these RNA sequences once transcribed, which for SEQ ID No. 6-11 above would be:

| (SEQ ID No. 31) | |
|---|---|
| | TGACTAG |
| (SEQ ID No. 32) | |
| | TAGCTAG |
| (SEQ ID No. 33) | |
| | TAACTAG |
| (SEQ ID NO. 34) | |
| | TGACAATTA |
| (SEQ ID No. 35) | |
| | TAGCAATTA |
| (SEQ ID No. 36) | |
| | TAACAATTA |

In particular a DNA sequence encoding a TRM may have the sequence shown as SEQ ID No. 37, which codes for STOP; Leucine; Alanine

| (SEQ ID NO. 37) | |
|---|---|
| | TGACTAGCA |

Where a nucleic acid construct comprises more than two transgenes, compound translational readthrough motifs may be placed in series, 5′ for sequences encoding cleavage sites. This enables multiple transgenes to be expressed at different ratios. With each additional readthrough motif the level of expression should be reduced 10 to 50-fold relative to the upstream transgene.

The nucleic acid sequence encoding IL-12 may be positioned downstream of a frame-slip motif (FSM) or a translational readthrough motif (TRM). The nucleic acid construct may have the general structure:

CAR-FSM/TRM-coexpr-IL12

in which:

“CAR” is a nucleic acid sequence encoding a chimeric antigen receptor

“FSM/TRM” is a frame-slip motif or a translational readthrough motif

“coexpr” is a sequence enabling the co-expression of the CAR and IL-12 as separate polypeptides; and

“IL-12” is a nucleic acid sequence encoding IL-12 or flexi-IL12

In constructs which comprise a nucleic acid sequence encoding another cytokine or chemokine or a chemokine, this may also be placed downstream of the FSM or TRM, such that the level of expression of the other cytokine(s) or chemokine(s) is also reduced (compared to the level of expression which would be obtained in the absence of the FSM or TRM sequence). In this respect, the level of expression of the other cytokine(s) or chemokine(s) may be reduced comparable to that of IL-12. Such a construct may have the general structure:

CAR-FSM/TRM-coexpr1-IL12-coexpr2-CC; or

CAR-FSM/TRM-coexpr1-CC-coexpr2-IL12

in which:

“CAR” is a nucleic acid sequence encoding a chimeric antigen receptor

“FSM/TRM” is a frame-slip motif or a translational readthrough motif

“coexpr1” and “coexpr2”, which may be the same or different, are sequence enabling the co-expression of the CAR, IL-12 and the cytokine or chemokine as separate polypeptides;

“IL-12” is a nucleic acid sequence encoding IL-12 or flexi-IL12; and

“CC” is a nucleic acid sequence encoding a cytokine (other than IL-12) or a chemokine.

Alternatively, the nucleic acid sequence encoding the other cytokine or encoding a chemokine may be placed upstream of the FSM or TRM or on a separate construct, such that its expression is unaffected by the FSM or TRM. With such an arrangement, the level of expression of other cytokine(s) or chemokine(s) will exceed the level of expression of 11-12. Such a construct may have the general structure:

CAR-coexpr1-CC-FSM/TRM-coexpr2-IL12; or

CC-coexpr1-CAR-FSM/TRM-coexpr2-IL12

in which:

“CAR” is a nucleic acid sequence encoding a chimeric antigen receptor

“FSM/TRM” is a frame-slip motif or a translational readthrough motif

“coexpr1” and “coexpr2”, which may be the same or different, are sequence enabling the co-expression of the CAR, IL-12 and the cytokine or chemokine as separate polypeptides;

“IL-12” is a nucleic acid sequence encoding IL-12 or flexi-IL12; and

“CC” is a nucleic acid sequence encoding a cytokine (other than IL-12) or a chemokine.

A third option is the nucleic acid sequence encoding other cytokine(s) or chemokine(s) may be placed downstream of a FSM or TRM which is different to the one upstream of the IL-12 encoding sequence. This may have “compound” effects. For example, a nucleic acid construct with compound translational readthrough motifs may have the structure:

CAR-FSM/TRM1-coexpr1-CC-FSM/TRM2-coexpr2-IL12

in which:

“CAR” is a nucleic acid sequence encoding a chimeric antigen receptor

“FSM/TRM1” and “FSM/TRM2”, which may be the same of different is/are frame-slip motif(s) or translational readthrough motif(s);

“coexpr1” and “coexpr2”, which may be the same or different, are sequence enabling the co-expression of the CAR, IL-12 and the cytokine or chemokine as separate polypeptides;

“IL-12” is a nucleic acid sequence encoding IL-12 or flexi-IL12; and

“CC” is a nucleic acid sequence encoding a cytokine (other than IL-12) or a chemokine.

In this arrangement, the level of expression of CAR will be higher than the level of expression of the cytokine/chemokine, which will be higher than the level of expression of IL-12.

Altered Signal Peptides

While translational readthrough motifs significantly decrease transgene expression, in certain situations it might be necessary to reduce the expression levels even further. For secreted proteins a further reduction in expression can be achieved by combining a translational readthrough motif with an altered signal peptide sequence. In this case, the altered signal peptide and second transgene are placed 3′ of the translational readthrough motif and self-cleaving peptide sequence (FIG. 3D).

A signal peptide is a short peptide, commonly 5-30 amino acids long, present at the N-terminus of the majority of newly synthesized proteins that are destined towards the secretory pathway. These proteins include those that reside either inside certain organelles (for example, the endoplasmic reticulum, golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

Altered signal peptides are described in detail in WO2016/174409, which is herein incorporated by reference. The altered signal peptide may comprise one or more mutation(s), such as substitutions or deletions, such that it has fewer hydrophobic amino acids than the wild-type signal peptide from which it is derived. The term “wild type” means the sequence of the signal peptide which occurs in the natural protein from which it is derived.

Where the nucleic acid construct comprises two transgenes both encoding transmembrane proteins, the protein encoded by the downstream transgene (which has lower relative expression) may comprise fewer hydrophobic amino acids than the protein encoded by the upstream transgene (which has higher relative expression).

The hydrophobic amino acids mutated in order to alter signal peptide efficiency may be: Alanine (A); Valine (V); Isoleucine (I); Leucine (L); Methionine (M); Phenylalanine (P); Tyrosine (Y); or Tryptophan (W).

The altered signal peptide may comprise 1, 2, 3, 4 or 5 amino acid deletions or substitutions of hydrophobic amino acids. Hydrophobic amino acids may be replaced with non-hydrophobic amino acids, such as hydrophilic or neutral amino acids.

Cleavage Site

The invention provides a nucleic acid construct comprising:
(i) a nucleic acid sequence encoding a chimeric antigen receptor (CAR); and
(ii) a nucleic acid sequence encoding interleukin 12 (IL-12) downstream of an FSM/TRM.

The nucleic acid construct may also comprise one or more of the following:
(iii) a nucleic acid sequence encoding a dominant negative SHP2;
(iv) a nucleic acid sequence encoding a dominant negative TGFβ receptor;
(v) a nucleic acid sequence encoding a chimeric cytokine receptor:
(vi) one or more nucleic acid sequence(s) encoding one or more additional cytokine(s) or chemokine(s).

In order that the CAR and IL-12 and optionally one or more additional components may be expressed as separate polypeptides, the construct may comprise a sequence encoding a cleavage site positioned between nucleic acid sequences which encode the two polypeptides.

The cleavage site may be any sequence which enables the polypeptide produced by translation of the nucleic acid construct to split or become separated.

The term “cleavage” is used herein for convenience, but the cleavage site may cause two polypeptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode first and second polypeptides, causes the first and second polypeptides to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′ (SEQ ID No. 38)) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (SEQ ID No. 39) (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the first and second polypeptides and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus.

The C-terminal 19 amino acids of the longer cardiovirus protein, together with the N-terminal proline of 2B mediate “cleavage” with an efficiency approximately equal to the apthovirus FMDV 2a sequence. Cardioviruses include encephalomyocarditis virus (EMCV) and Theiler's murine encephalitis virus (TMEV).

Mutational analysis of EMCV and FMDV 2A has revealed that the motif DxExNPGP (SEQ ID No. 40) is intimately involved in “cleavage” activity (Donelly et al (2001) as above).

The cleavage site of the present invention may comprise the amino acid sequence:

Dx₁Ex₂NPGP, where x₁ and x₂ are any amino acid. X₁ may be selected from the following group: I, V, M and S. X₂ may be selected from the following group: T, M, S, L, E, Q and F.

For example, the cleavage site may comprise one of the amino acid sequences shown in Table 1.

TABLE 1

| Motif | Present in: |
|---|---|
| DIETNPGP | Picornaviruses EMCB, |
| (SEQ ID No. 41) | EMCD, EMCPV21 |
| DVETNPGP | Picornaviruses MENGO and |
| (SEQ ID No. 42) | TMEBEAN; Insect virus |
| | DCV, ABPV |
| DVEMNPGP | Picornaviruses TMEGD7 and |
| (SEQ ID No. 43) | TMEBEAN |
| DVESNPGP | Picornaviruses FMDA10, |
| (SEQ ID No. 44) | FMDA12, FMDC1, FMD01K, |
| | FMDSAT3, FMDVSAT2, |
| | ERAV; Insect virus CrPV |
| DMESNPGP | Picornavirus FMDV01G |
| (SEQ ID No. 45) | |
| DVELNPGP | Picornavirus ERBV; Porcine |
| (SEQ ID No. 45) | rotavirus |
| DVEENPGP | Picornavirus PTV-1; Insect |
| (SEQ ID No. 46) | virus TaV; Trypanosoma |
| | TSR1 |
| DIELNPGP | Bovine Rotavirus, human |
| (SEQ ID No. 47) | rotavirus |
| DIEQNPGP | Trypanosoma AP |
| (SEQ ID No. 48) | endonuclease |
| DSEFNPGP | Bacterial sequence T. |
| (SEQ ID No. 49) | maritima |

The cleavage site, based on a 2A sequence may be, for example 15-22 amino acids in length. The sequence may comprise the C-terminus of a 2A protein, followed by a proline residue (which corresponds to the N-terminal proline of 2B).

Mutational studies have also shown that, in addition to the naturally occurring 2A sequences, some variants are also active. The cleavage site may correspond to a variant sequence from a naturally occurring 2A polypeptide, have one, two or three amino acid substitutions, which retains the capacity to induce the “cleavage” of a polyprotein sequence into two or more separate proteins.

The cleavage sequence may be selected from the following which have all been shown to be active to a certain extent (Donnelly et al (2001) as above):

(SEQ ID No. 50)
LLNFDLLKLAGDVESNPGP
(SEQ ID No. 51)
LLNFDLLKLAGDVQSNPGP
(SEQ ID No. 52)
LLNFDLLKLAGDVEINPGP
(SEQ ID No. 53)
LLNFDLLKLAGDVEFNPGP
(SEQ ID No. 54)
LLNFDLLKLAGDVESHPGP
(SEQ ID No. 55)
LLNFDLLKLAGDVESEPGP
(SEQ ID No. 56)
LLNFDLLKLAGDVESQPGP
(SEQ ID NO. 57)
LLNFDLLKLAGDVESNPGG

Based on the sequence of the DxExNPGP “a motif, “2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

(SEQ ID No. 58)
YHADYYKQRLIHDVEMNPGP
(SEQ ID No. 59)
HYAGYFADLLIHDIETNPGP
(SEQ ID No. 60)
QCTNYALLKLAGDVESNPGP
(SEQ ID No. 61)
ATNFSLLKQAGDVEENPGP
(SEQ ID No. 62)
AARQMLLLLSGDVETNPGP
(SEQ ID No. 63)
RAEGRGSLLTCGDVEENPGP
(SEQ ID No. 64)
TRAEIEDELIRAGIESNPGP
(SEQ ID No. 65)
TRAEIEDELIRADIESNPGP
(SEQ ID NO. 66)
AKFQIDKILISGDVELNPGP
(SEQ ID No. 67)
SSIIRTKMLVSGDVEENPGP
(SEQ ID No. 68)
CDAQRQKLLLSGDIEQNPGP
(SEQ ID No. 69)
YPIDFGGFLVKADSEFNPGP

The cleavage site may comprise the 2A-like sequence shown as SEQ ID No. 630(RAEGRGSLLTCGDVEENPGP) or 2A peptide from Thosea asigna virus capsid protein, which has the sequence EGRGSLLTCGDVEENPGP (SEQ ID No. 70) It has been shown that including an N-terminal “extension” of between 5 and 39 amino acids can increase activity (Donnelly et al (2001) as above). In particular, the cleavage sequence may comprise one of the following sequences or a variant thereof having, for example, up to 5 amino acid changes which retains cleavage site activity:

(SEQ ID No. 71)
VTELLYRMKRAETYCPRPLAIHPTEARHKQKIVAPVKQTLNFDLLKLAGD
VESNPGP
(SEQ ID No. 72)
LLAIHPTEARHKQKIVAPVKQTLNFDLLKLAGDVESNPGP
(SEQ ID No. 73)
EARHKQKIVAPVKQTLNFDLLKLAGDVESNPGP
(SEQ ID NO. 74)
APVKQTLNFDLLKLAGDVESNPGP

Chimeric Antigen Receptors (CARS)

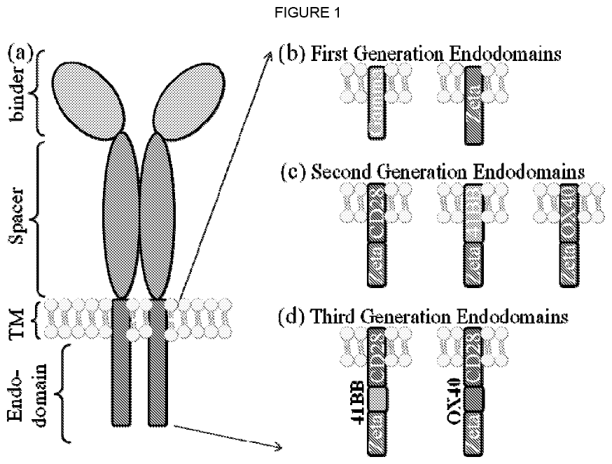

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3 results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral or lentiviral vectors to generate cancer-specific T cells for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

Bispecific CARs, known as tandem CARs or TanCARs, have been developed to target two or more cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. When a TanCAR binds either or both of the target antigens, this results in the transmission of an activating signal to the cell on which it is expressed.

Antigen Binding Domain

The antigen binding domain is the portion of CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

In a classical CAR, the antigen-binding domain comprises: a single-chain variable fragment (scFv) derived from a monoclonal antibody. CARs have also been produced with domain antibody (dAb) or VHH antigen binding domains or which comprise a Fab fragment of, for example, a monoclonal antibody. A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The two chains of a Fab CAR may have the general structure:

VH-CH-spacer-transmembrane domain-intracellular signalling domain; and

VL-CL

or

VL-CL-spacer-transmembrane domain-intracellular signalling domain; and

VH-CH

For Fab-type chimeric receptors, the antigen binding domain is made up of a VH from one polypeptide chain and a VL from another polypeptide chain.

The polypeptide chains may comprise a linker between the VH/VL domain and the

CH/CL domains. The linker may be flexible and serve to spatially separate the VH/VL domain from the CH/CL domain.

The antigen-binding domain of the CAR may bind a tumour associated antigen. Various tumour associated antigens (TAA) are known, for example as shown in the following Table 2.

TABLE 2

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The CAR may bind one of the targets summarised in Table 3 below. In particular, the CAR may bind one of the following target antigens: disialoganglioside (GD2); epidermal growth factor receptor (EGFR), Epithelial cell adhesion molecule (EpCAM), Glypican 3 (GPC3), human epidermal growth factor receptor (HER2), L1CAM, Mucin 1 (MUC1), or Prostate-specific membrane antigen (PSMA).

In one embodiment of the invention the CAR may bind an antigen other than PSMA. The CAR may bind one of the target antigens listed in Table 3 which is not PSMA. For example, the CAR may bind one of the following target antigens: disialoganglioside (GD2); epidermal growth factor receptor (EGFR), Epithelial cell adhesion molecule (EpCAM), Glypican 3 (GPC3), human epidermal growth factor receptor (HER2), L1CAM and Mucin 1 (MUC1).

Disialoganglioside GD2

CARs have been developed which bind disialoganglioside (GD2) a sialic acid-containing glycosphinolipid. Such CARs may, for example, be based on the GD2 binder 14g2a, or huK666 as described in WO2015/132604.

A CAR which binds GD2 may have an antigen-binding domain which comprises:

a) a heavy chain variable region (VH) having complementarity determining regions (CDRs) with the following sequences:

| (SEQ ID No. 12) | |
|---|---|
| CDR1 - | SYNIH; |
| (SEQ ID No. 13) | |
| CDR2 - | VIWAGGSTNYNSALMS |
| (SEQ ID No. 14) | |
| CDR3 - | RSDDYSWFAY; |

and

b) a light chain variable region (VL) having CDRs with the following sequences:

| (SEQ ID No. 15) | |
|---|---|
| CDR1 - | RASSSVSSSYLH; |
| (SEQ ID No. 16) | |
| CDR2 - | STSNLAS |
| (SEQ ID No. 17) | |
| CDR3 - | QQYSGYPIT. |

The GD2 binding domain may comprise a VH domain having the sequence shown as SEQ ID No. 75; and/or a VL domain having the sequence shown as SEQ ID No 76.

(Humanised KM666 VH sequence)
SEQ ID No. 75
QVQLQESGPGLVKPSQTLSITCTVSGFSLASYNIHWVRQPPGKGLEWLGV
IWAGGSTNYNSALMSRLTISKDNSKNQVFLKMSSLTAADTAVYYCAKRSD
DYSWFAYWGQGTLVTVSS
(Humanised KM666 VL sequence)
SEQ ID No. 76
ENQMTQSPSSLSASVGDRVTMTCRASSSVSSSYLHWYQQKSGKAPKVWIY
STSNLASGVPSRFSGSGSGTDYTLTISSLQPEDFATYYCQQYSGYPITFG
QGTKVEIK

The antigen-binding domain of the CAR may comprise a variant of SEQ ID NO: 75 or 76 having at least 80, 85, 90, 95, 98 or 99% sequence identity, provided that the variant sequence retains the capacity to bind GD2.

Epidermal Growth Factor Receptor (EGFR)

Epidermal growth factor receptor (EGFR; ErbB-1; HER1 in humans) is a transmembrane protein that is a receptor for members of the epidermal growth factor family (EGF family) of extracellular protein ligands. The amino acid sequence for human EpCAM is available from UniProt, Accession No. P00533. EGFR undergoes a transition from an inactive monomeric form to an active homodimer. EGFR dimerization stimulates its intrinsic intracellular protein-tyrosine kinase activity, causing autophosphorylation of several tyrosine (Y) residues in the C-terminal domain, including Y992, Y1045, Y1068, Y1148 and Y1173. Autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation.

Over-expression of EGFR is associated with the development of a wide variety of tumours. Mutations that lead to EGFR overexpression (known as upregulation or amplification) have been associated with a number of cancers, including adenocarcinoma of the lung (40% of cases), anal cancers, glioblastoma (50%) and epithelial tumors of the head and neck (80-100%). These somatic mutations involving EGFR lead to its constant activation, which produces uncontrolled cell division. In glioblastoma a specific mutation of EGFR, called EGFRvIII, is often observed. Mutations, amplifications or misregulations of EGFR or family members are implicated in about 30% of all epithelial cancers.

Several anti-EGFR antibodies are known, for example, cetuximab, panitumumab, zalutumumab, nimotuzumab, and matuzumab.

Epithelial Cell Adhesion Molecule (EpCAM)

Epithelial cell adhesion molecule (EpCAM) is a transmembrane glycoprotein mediating Ca2+-independent homotypic cell—cell adhesion in epithelia. EpCAM is also involved in cell signaling, migration, proliferation, and differentiation. Additionally, EpCAM has oncogenic potential via its capacity to upregulate c-myc, e-fabp, and cyclins A & E. Since EpCAM is expressed exclusively in epithelia and epithelial-derived neoplasms, EpCAM can be used as diagnostic marker for various cancers. It appears to play a role in tumorigenesis and metastasis of carcinomas, so it can also act as a prognostic marker and as a target for immunotherapeutic strategies. EpCAM is often overexpressed in certain carcinomas, including in breast cancer, colon cancer and basal cell carcinoma of the skin.

EpCAM is a glycosylated, 30- to 40-kDa type I membrane protein. The amino acid sequence for human EpCAM is available from UniProt, Accession No. P16422. EpCAM can be cleaved which lends the molecule oncogenic potential. Upon cleavage, the extracellular domain (EpEX) is released into the area surrounding the cell, and the intracellular domain (EpICD) is released into the cytoplasm of the cell. EpICD forms a complex with the proteins FHL2, β-catenin, and Lef inside the nucleus. This complex then binds to DNA and promotes the transcription of various genes. Targets of upregulation include c-myc, e-fabp, and cyclins A & E. This has the effect of promoting tumour growth. Additionally, EpEX that has been cleaved can stimulate the cleavage of additional EpCAM molecules resulting in a positive feedback loop. EpCAM may also play a role in epithelial mesenchymal transition (EMT) in tumours.

Since EpCAM is expressed mostly on the basolateral membrane in normal epithelia, it should be much less accessible to antibodies than EpCAM in cancer tissue, where it is homogeneously distributed on the cancer cell surface. In addition to being overexpressed in many carcinomas, EpCAM is expressed in cancer stem cells, making EpCAM an attractive target for immunotherapy. However, the heterogeneous expression of EpCAM in carcinomas and the fact that EpCAM is not tumor-specific (i.e., it is found in normal epithelium) has hampered development of immunotherapy approaches directed towards EpCAM to date.

Several anti-EpCAM antibodies are known including murine IgG2α edrecolomab and its murine/human chimeric IgG1 antibody version, and humanized, human-engineered and fully human IgG1 antibodies 3622W94, ING-1, and adecatumumab (MT201), respectively.

Glypican 3 (GPC3)

Glypican-3 plays a role in the control of cell division and growth regulation. The protein core of GPC3 consists of two subunits, where the N-terminal subunit has a size of ˜40 kDa and the C-terminal subunit is ˜30 kDa. The amino acid sequence for human GPC3 is available from UniProt, Accession No. P51654.

Six glypicans (GPC1-6) have been identified in mammals. Cell surface heparan sulfate proteoglycans are composed of a membrane-associated protein core substituted with a variable number of heparan sulfate chains. Members of the glypican-related integral membrane proteoglycan family (GRIPS) contain a core protein anchored to the cytoplasmic membrane via a glycosyl phosphatidylinositol linkage. GPC3 interacts with both Wnt and frizzled (FZD) to form a complex and trigger downstream signalling.

GPC3 is a promising therapeutic target for treating liver cancer. Several therapeutic anti-GPC3 antibodies have been developed, including GC33, HN3 and YP7. YP7 and its humanised form hYP7 binds the C-lobe of GPC3; the single-domain antibody, HN3, targets the N-lobe of GPC3; whereas the human monoclonal antibody targets the heparan sulfate moiety of GPC3. Chimeric antigen receptor (CAR) T cell immunotherapies based on GC33, hYP7 and HN3 are being developed at various stages for treating liver cancer.

Human Epidermal Growth Factor Receptor (HER2)

HER2 (from human epidermal growth factor receptor 2) is also known as HER2/neu, Receptor tyrosine-protein kinase erbB-2, CD340 (cluster of differentiation 340), proto-oncogene Neu, Erbb2 (rodent), and ERBB2 (human.

HER2 is a member of the human epidermal growth factor receptor (HER/EGFR/ERBB) family. Amplification or over-expression of this oncogene has been shown to play an important role in the development and progression of certain aggressive types of breast cancer. In recent years the protein has become an important biomarker and target of therapy for approximately 30% of breast cancer patients.

The ErbB family consists of four plasma membrane-bound receptor tyrosine kinases. One of which is erbB-2, and the other members being epidermal growth factor receptor, erbB-3 (neuregulin-binding; lacks kinase domain), and erbB-4. All four contain an extracellular ligand binding domain, a transmembrane domain, and an intracellular domain that can interact with a multitude of signaling molecules and exhibit both ligand-dependent and ligand-independent activity. HER2 can heterodimerise with any of the other three receptors and is considered to be the preferred dimerisation partner of the other ErbB receptors. Dimerisation results in the autophosphorylation of tyrosine residues within the cytoplasmic domain of the receptors and initiates a variety of signaling pathways. The amino acid sequence for human HER2 is available from UniProt, Accession No. P04626.

HER2 is the target of the monoclonal antibody trastuzumab (marketed as Herceptin). Trastuzumab is effective only in cancers where HER2 is over-expressed. An important downstream effect of trastuzumab binding to HER2 is an increase in p27, a protein that halts cell proliferation. Another monoclonal antibody, Pertuzumab, which inhibits dimerisation of HER2 and HERS receptors, was approved by the FDA for use in combination with trastuzumab in June 2012.

L1 Cell Adhesion Molecule (L1CAM) L1CAM, also known as L1, is a transmembrane protein member of the L1 protein family, encoded by the L1CAM gene. This protein, of 200-220 kDa, is formed of six immunoglobulin domains followed by five fibronectin type III domains which are connected to a small intracellular domain by a transmembrane helix. The amino acid sequence for human L1CAM is available from UniProt, Accession No. P32004. L1CAM is a neuronal cell adhesion molecule with a strong implication in cell migration, adhesion, neurite outgrowth, myelination and neuronal differentiation. L1CAM is located all over the nervous system on the surface of neurons. It is placed along the cellular membrane so that one end of the protein remains inside the nerve cell while the other end stays on the outer surface of the neurone. This position allows the protein to activate chemical signals which spread through the neurone.

A wide variety of cells express L1CAM, including immature oligodendrocytes and Schwann cells, which are non-neuronal cells that provide support and protection for neurons and form myelin; T cells which are lymphocytes involved in cell-mediated immunity; other types of lymphocytes such as B cells and monocytes. L1CAM is expressed in multiple tumour types, for example melanoma and lung carcinoma cells.

Wolterink et al (2010) Cancer Res 15:2504-2515 describe the generation a series of novel monoclonal antibodies (mAb) to the L1CAM ectodomain, including the antibody L1-9.3.

Mucin 1 (MUC1)

Mucin 1, cell surface associated (MUC1), also called polymorphic epithelial mucin (PEM) or epithelial membrane antigen or EMA, is a mucin encoded by the MUC1 gene in humans. MUC1 is a glycoprotein with extensive O-linked glycosylation of its extracellular domain. Mucins line the apical surface of epithelial cells in the lungs, stomach, intestines, eyes and several other organs. Mucins protect the body from infection by pathogen binding to oligosaccharides in the extracellular domain, preventing the pathogen from reaching the cell surface. Overexpression of MUC1 is often associated with colon, breast, ovarian, lung and pancreatic cancers. The amino acid sequence for human MUC1 is available from UniProt, Accession No. P15941.

Numerous anti-mucin 1 (anti-MUC1) antibodies have been developed and characterised, including 1 B2 and 12D10 which recognise specific O-glycan structures at the PDT*R motif (the asterisk represents an O-glycosylation site). 1B2 recognizes O-glycans with an unsubstituted O-6 position of the GaINAc residue (Tn, T, and 23ST), whereas 12D10 recognizes Neu5Ac at the same position (STn, 26ST, and dST).

Prostate-Specific Membrane Antigen (PSMA)

Prostate-specific membrane antigen (PSMA), also known as glutamate carboxypeptidase II (GCPII), N-acetyl-L-aspartyl-L-glutamate peptidase I (NAALADase I) or NAAG peptidase, is a zinc metalloenzyme. It is a class II membrane glycoprotein that catalyzes the hydrolysis of N-acetylaspartylglutamate (NAAG) to glutamate and N-acetylaspartate (NAA). The amino acid sequence for human MUC1 is available from UniProt, Accession No. Q04609.

Human PSMA is highly expressed in the prostate, roughly a hundred times greater than in most other tissues. In some prostate cancers, PSMA is the second-most upregulated gene product, with an 8- to 12-fold increase over levels in noncancerous prostate cells. Because of this high expression, PSMA is being developed as potential biomarker for therapy and imaging of some cancers. In human prostate cancer, the higher expressing tumours are associated with quicker time to progression and a greater percentage of patients suffering relapse. In vitro studies using prostate and breast cancer cell lines with decreased PSMA levels showed a significant decrease in the proliferation, migration, invasion, adhesion and survival of the cells.

T-cells expressing CARs specific for prostate-specific membrane antigen (PSMA) are currenty in clinical trial for the treatment of prostate cancer (Junhans et al (2016) Prostate 76:1257-1270).

United Kingdom patent application No. 1919019.8 described various PSMA antigen-binding domains suitable for use in CARs.

A CAR which binds PSMA may have an antigen-binding domain which comprises:

a) a heavy chain variable region (VH) having complementarity determining regions (CDRs) with the following sequences:

| (SEQ ID No. 18) | |
|---|---|
| CDR1 - | SSWMN; |
| (SEQ ID NO. 19) | |
| CDR2 - | RIYPGDGDTNYAQKFQG |
| (SEQ ID No. 20) | |
| CDR3 - | GTGYLWYFDV; |

and

b) a light chain variable region (VL) having CDRs with the following sequences:

| (SEQ ID No. 21) | |
|---|---|
| CDR1 - | RASQDINENLA; |
| (SEQ ID No. 22) | |
| CDR2 - | YTSNRAT |
| (SEQ ID NO. 23) | |
| CDR3 - | QQYDNLPFT. |

The PSMA binding domain may comprise a VH domain having the sequence shown as SEQ ID No. 77; and/or a VL domain having the sequence shown as SEQ ID No 78.

(Humanised 7A12 VH sequence)
SEQ ID No. 77
QVQLVQSGAEVKKPGASVKVSCKASGYTFTSSWMNWVRQAPGQGLEWMGR
IYPGDGDTNYAQKFQGRVTMTRDTSISTAYMELSRLRSDDTAVYYCARGT
GYLWYFDVWGQGTMVTVSS
(Humanised 7A12 VL sequence)
SEQ ID No. 78
EIVLTQSPATLSLSPGERATLSCRASQDINENLAWYQQKPGQAPRLLIYY
TSNRATGIPARFSGSGSGRDFTLTISSLEPEDFAVYYCQQYDNLPFTFGQ
GTKVEIKR

The antigen-binding domain of the CAR may comprise a variant of SEQ ID NO: 77 or 78 having at least 80, 85, 90, 95, 98 or 99% sequence identity, provided that the variant sequence retains the capacity to bind PSMA.

In one embodiment of the invention, however, the CAR binds to an antigen other than PSMA. The CAR may have a non-PSMA specific antigen binding domain.

Intracellular T Cell Signaling Domain (Endodomain)

The CAR may comprise or associate with an activating endodomain: the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR may comprise the CD28 endodomain and CD3-Zeta endodomain. The sequences for these endodomains are shown below as SEQ ID Nos. 79 and 80 respectively:

(CD28 endodomain)
SEQ ID No. 79
RSKRSRLLHSDYMNMTPRRPGPTRKHYQPYAPPRDFAAYRS
(CD3z endodomain)
SEQ ID No. 80
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The endodomain may comprise:

(i) an ITAM-containing endodomain, such as the endodomain from CD3 zeta; and/or

(ii) a co-stimulatory domain, such as the endodomain from CD28; and/or

(iii) a domain which transmits a survival signal, for example a TNF receptor family endodomain such as OX-40 or 4-1BB.

An endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I (SeQ ID NO. 81). Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

A number of systems have been described in which the antigen recognition portion of the CAR is on a separate molecule from the signal transmission portion, such as those described in WO015/150771; WO2016/124930 and WO2016/030691. One or more of the viral vectors used in the method of the invention may encode such a “split CAR”. Alternatively one vector may comprise a nucleic acid sequence encoding the antigen recognition portion and one vector may comprise a nucleic acid sequence encoding the intracellular signalling domain.

Signal Peptide

One or more nucleic acid sequences in the vector may encode a signal peptide so that when the CAR, cytokine or chemokine is expressed inside a cell, the nascent protein is directed to the endoplasmic reticulum and subsequently to the cell surface, where it is expressed (or secreted in the case of the cytokine or chemokine).

The core of the signal peptide may contain a long stretch of hydrophobic amino acids that tends to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

A CAR may have the general formula:

Signal peptide-antigen binding domain-spacer domain-transmembrane domain-intracellular T cell signaling domain (endodomain).

Spacer

The CAR may comprise a spacer sequence to connect the antigen binding domain with the transmembrane domain and spatially separate the antigen binding domain from the endodomain. A flexible spacer allows to the antigen binding domain to orient in different directions to enable antigen binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

Where the composition of viral vectors includes more than one vector comprising a nucleic acid sequence encoding a CAR, the CARs may have different spacers.

OR Gates

A cell composition of the present invention may comprise two or more CARs. This may be as a result of transduction with two or more vectors, each comprising a nucleic acid sequence encoding a CAR; or it may be as a result of transduction with a single vector which comprises a nucleic acid construct encoding two or more CARs.

A CAR may be used in a combination with one or more other activatory or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:

“OR GATE”— T cell triggers when either antigen A or antigen B is present on the target cell

“AND GATE”— T cell triggers only when both antigens A and B are present on the target cell

“AND NOT GATE”— T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A+antigen B. This is especially important for antigens expressed at variable or low density on the target cell, as the level of a single antigen may be below the threshold needed for effective targeting by a CAR-T cell. Also, it avoids the phenomenon of antigen escape.

Transgenic T-Cell Receptor (TCR)

The cell of the present invention may express a transgenic TCR.

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (a) chain and a beta (β) chain (encoded by TRA and TRB, respectively), whereas in 5% of T cells the TCR consists of gamma and delta (γ/β) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example, the genes for engineered TCRs may be introduced into autologous T cells and transferred back into patients for T cell adoptive therapies. Such ‘heterologous’ TCRs may also be referred to herein as ‘transgenic TCRs’ or engineered TCRs.

The cell of the present invention may comprise one or more heterologous nucleic acid sequence(s) encoding a TRA and a TRB gene; or one or more heterologous nucleic acid sequence(s) encoding a TRG and a TRD gene.

Dominant Negative SHP-2

The cell of the present invention may express a dominant negative SHP-2.

WO2016/193696 describes truncated versions of SHP-1 and SHP-2 which block or reduces the inhibition mediated by inhibitory immunoreceptors such as CTLA4, PD-1, LAG-3, 2B4 or BTLA 1. The truncated forms of SHP-1 or SHP-2 comprise one or both SH2 domains, but lacks the phosphatase domain. When expressed in a CAR-T cell, these molecules act as dominant negative versions of wild-type SHP-1 and SHP-2 and compete with the endogenous molecule for binding to phosphorylated ITIMs.

The cell of the present invention may express a truncated protein which comprises an SH2 domain from a protein which binds a phosphorylated immunoreceptor tyrosine-based inhibition motif (ITIM) but lacks a phosphatase domain. The truncated protein may comprise one or both SHP-1 SH2 domain(s) but lack the SHP-1 phosphatase domain. Alternatively the truncated protein may comprise one or both SHP-2 SH2 domain(s) but lack the SHP-2 phosphatase domain.

SHP-1

Src homology region 2 domain-containing phosphatase-1 (SHP-1) is a member of the protein tyrosine phosphatase family. It is also known as PTPN6.

The N-terminal region of SHP-1 contains two tandem SH2 domains which mediate the interaction of SHP-1 and its substrates. The C-terminal region contains a tyrosine-protein phosphatase domain.

SHP-1 is capable of binding to, and propagating signals from, a number of inhibitory immune receptors or ITIM containing receptors. Examples of such receptors include, but are not limited to, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

An activity modulator may comprise or consist of the SHP-1 tandem SH2 domain which is shown below as SEQ ID NO: 82.

SHP-1 SH2 complete domain
(SEQ ID NO: 82)
MVRWFHRDLSGLDAETLLKGRGVHGSFLARPSRKNQGDFSLSVRVGDQVT
HIRIQNSGDFYDLYGGEKFATLTELVEYYTQQQGVLQDRDGTIIHLKYPL
NCSDPTSERWYHGHMSGGQAETLLQAKGEPWTFLVRESLSQPGDFVLSVL
SDQPKAGPGSPLRVTHIKVMCEGGRYTVGGLETFDSLTDLVEHFKKTGIE
EASGAFVYLRQPYY

SHP-1 has two SH2 domains at the N-terminal end of the sequence, at residues 4-100 and 110-213. An activity modulator may comprise one or both of the sequences shown as SEQ ID No. 83 and 84.

SHP-1 SH2 1
(SEQ ID NO: 83)
WFHRDLSGLDAETLLKGRGVHGSFLARPSRKNQGDFSLSVRVGDQVTHIR
IQNSGDFYDLYGGEKFATLTELVEYYTQQQGVLQDRDGTIIHLKYPL
SHP-1 SH2 2
(SEQ ID No. 84)
WYHGHMSGGQAETLLQAKGEPWTFLVRESLSQPGDFVLSVLSDQPKAGPG
SPLRVTHIKVMCEGGRYTVGGLETFDSLTDLVEHFKKTGIEEASGAFVYL
RQPY

The cell may express a variant of SEQ ID NO: 82, 83 or 84 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-1.

SHP-2

SHP-2, also known as PTPN11, PTP-1D and PTP-2C is is a member of the protein tyrosine phosphatase (PTP) family. Like PTPN6, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

An activity modulator may comprise or consist of the SHP-1 tandem SH2 domain which is shown below as SEQ ID NO: 87. SHP-1 has two SH2 domains at the N-terminal end of the sequence, at residues 6-102 and 112-216. An activity modulator may comprise one or both of the sequences shown as SEQ ID No. 85 and 86.

SHP-2 first SH2 domain
(SEQ ID NO: 85)
WFHGHLSGKEAEKLLTEKGKHGSFLVRESQSHPGDFVLSVRTGDDKGESN
DGKSKYDLYGGEKFATLAELVQYYMEHHGQLKEKNGDVIELKYPL
SHP-2 second SH2 domain
(SEQ ID No. 86)
WFHGHLSGKEAEKLLTEKGKHGSFLVRESQSHPGDFVLSVRTGDDKGESN
DGKSKVTHVMIRCQELKYDVGGGERFDSLTDLVEHYKKNPMVETLGTVLQ
LKQPL
SHP-2 both SH2 domains
(SEQ ID No. 87)
WFHPNITGVEAENLLLTRGVDGSFLARPSKSNPGDFTLSVRRNGAVTHIK
IQNTGDYYDLYGGEKFATLAELVQYYMEHHGQLKEKNGDVIELKYPLNCA
DPTSERWFHGHLSGKEAEKLLTEKGKHGSFLVRESQSHPGDFVLSVRTGD
DKGESNDGKSKVTHVMIRCQELKYDVGGGERFDSLTDLVEHYKKNPMVET
LGTVLQLKQPL

The cell may express a variant of SEQ ID NO: 85, 86 or 87 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-2.

Dominant Negative TGFβ Receptor

The cell of the present invention may express a dominant negative TGFβ receptor.

Engineered cells face hostile microenvironments which limit adoptive immunotherapy. One of the main inhibitory mechanisms within the tumour microenvironment is transforming growth factor beta (TGFβ). The TGFβ signalling pathway has a pivotal role in the regulatory signalling that controls a variety of cellular processes. TGFβ play also a central role in T cell homeostasis and control of cellular function. Particularly, TGFβ signalling is linked to an immuno-depressed state of the T-cells, with reduced proliferation and activation. TGFβ expression is associated with the immunosuppressive microenvironment of tumour.

A variety of cancerous tumour cells are known to produce TGFβ directly. In addition to the TGFβ production by cancerous cells, TGFβ can be produced by the wide variety of non-cancerous cells present at the tumour site such as tumour-associated T cells, natural killer (NK) cells, macrophages, epithelial cells and stromal cells.

The transforming growth factor beta receptors are a superfamily of serine/threonine kinase receptors. These receptors bind members of the TGFβ superfamily of growth factor and cytokine signalling proteins. There are five type II receptors (which are activatory receptors) and seven type I receptors (which are signalling propagating receptors).

Auxiliary co-receptors (also known as type III receptors) also exist. Each subfamily of the TGFβ superfamily of ligands binds to type I and type II receptors.

The three transforming growth factors have many activities. TGFβ1 and 2 are implicated in cancer, where they may stimulate the cancer stem cell, increase fibrosis/desmoplastic reactions and suppress immune recognition of the tumour.

The active TGFβ receptor (TβR) is a hetero-tetramer, composed by two TGFβ receptor I (T(βRI) and two TGFβ receptor II (T(βRII). TGFβ1 is secreted in a latent form and is activated by multiple mechanisms. Once activated it forms a complex with the TβRII TβRI that phosphorylates and activates WI.

The cell of the invention may express a dominant negative TGFβ receptor. A dominant negative TGFβ receptor may lack the kinase domain.

For example, the dominant negative TGFβ receptor may comprise or consist of the sequence shown as SEQ ID No. 88, which is a monomeric version of TGF receptor II

(dn TGFβ RII)
SEQ ID No. 88
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQVTGISL
LPPLGVAISVIIIFYCYRVNRQQKLSS

A dominant-negative TGF-βRII (dnTGF-βII) has been reported to enhance PSMA targeted CAR-T cell proliferation, cytokine secretion, resistance to exhaustion, long-term in vivo persistence, and the induction of tumour eradication in aggressive human prostate cancer mouse models (Kloss et al (2018) Mol. Ther. 26:1855-1866).

Chimeric Cytokine Receptor

The cell of the present invention may express a chimeric cytokine receptor. WO2017/029512 describes chimeric cytokine receptors (CCR) comprising: an exodomain which binds to a non-cytokine ligand; and a cytokine receptor endodomain.

The non-cytokine ligand may be selected from selected from a tumour secreted factor, a chemokine and a cell-surface antigen.

The chimeric cytokine receptor may comprise two polypeptides:
(i) a first polypeptide which comprises:
   (a) a first antigen-binding domain which binds a first epitope of the ligand
   (b) a first chain of the cytokine receptor endodomain; and
(ii) a second polypeptide which comprises:
   (a) a second antigen-binding domain which binds a second epitope of the ligand
   (b) a second chain of the cytokine-receptor endodomain.

Alternatively the chimeric cytokine receptor which comprises two polypeptides:
(i) a first polypeptide which comprises:
   (a) a heavy chain variable domain (VH)
   (b) a first chain of the cytokine receptor endodomain; and
(ii) a second polypeptide which comprises:
   (a) a light chain variable domain (VL)
   (b) a second chain of the cytokine-receptor endodomain.

For example, the cytokine receptor endodomain may comprise:

(i) IL-2 receptor β-chain endodomain

(ii) IL-7 receptor α-chain endodomain;

(iii) IL-15 receptor α-chain endodomain; or

(iv) common γ-chain receptor endodomain.

The cytokine receptor endodomain may comprise (i), (ii) or (iii); and (iv).

The cytokine receptor endodomain may comprise the α-chain endodomain and the β-chain endodomain from granulocyte-macrophage colony-stimulating factor receptor (GMCSF-R)

The ligand may be a tumour secreted factor, for example a tumour secreted factor selected from: prostate-specific antigen (PSA), carcinoembryonic antigen (CEA), vascular endothelial growth factor (VEGF) and CA125.

The ligand may be a chemokine, for example a chemokine selected from chemokine selected from: CXCL12, CCL2, CCL4, CCL5 and CCL22.

The ligand may be a cell-surface molecule, such as a transmembrane protein. The ligand may be, for example, CD22.

Constitutively Active Chimeric Cytokine Receptors

The cell of the invention may express a constitutively active chimeric cytokine receptor. A constitutively active chimeric cytokine receptor may comprise two chains which dimerise, either spontaneously or in the presence of an agent (a chemical inducer of dimerization or CID) bringing together two cytokine receptor endodomains.

The constitutively active chimeric cytokine receptor may therefore comprise a dimerization domain; and a cytokine receptor endodomain.

Dimerisation may occur spontaneously, in which case the chimeric transmembrane protein will be constitutively active. Alternatively, dimerization may occur only in the presence of a chemical inducer of dimerization (CID) in which case the transmembrane protein only causes cytokine-type signalling in the presence of the CID.

Suitable dimerization domains and CIDs are described in WO2015/150771, the contents of which are hereby incorporated by reference.

Where the dimerization domain spontaneously heterodimerizes, it may be based on the dimerization domain of an antibody. In particular it may comprise the dimerization portion of a heavy chain constant domain (CH) and a light chain constant domain (CL). The “dimerization portion” of a constant domain is the part of the sequence which forms the inter-chain disulphide bond.

The chimeric cytokine receptor may comprise the Fab portion of an antibody as exodomain. In this respect, the chimeric antigen may comprise two polypeptides:
(i) a first polypeptide which comprises:
   (a) a heavy chain constant domain (CH)
   (b) a first chain of the cytokine receptor endodomain; and
(ii) a second polypeptide which comprises:
   (a) a light chain constant domain (CL)
   (b) a second chain of the cytokine-receptor endodomain.

The cytokine receptor endodomain may comprise:
(i) IL-2 receptor β-chain endodomain
(ii) IL-7 receptor α-chain endodomain; or
(iii) IL-15 receptor α-chain endodomain; and/or
(iv) common γ-chain receptor endodomain.

The cytokine receptor endodomain may comprise the α-chain endodomain and the β-chain endodomain from granulocyte-macrophage colony-stimulating factor receptor (GMCSF-R)

A constitutively active CCR having an IL-2, IL-7 or GM-CSF receptor endodomain may have one of the following structures:

Fab_CCR_IL2: HuLightKappa-IL2RgTM-IL2RgEndo-2A-HuCH1-IL2bTM-IL2RbENDO Fab_CCR_IL7:

HuLightKappa-IL2RgTM-IL2RgEndo-2A-HuCH1-IL7RaTM-IL7RaENDO Fab_CCR_GMCSF:

HuLightKappa-GMCSFRbTM-GMCSFRbEndo-2A-HuCH1-GMCSFRaTM-GMCSFRaENDO

In which:

HuLightKappa is a human light kappa chain

IL2RgTM is a transmembrane domain from human IL2R common gamma chain

IL2RgEndo is an endodomain derived from human IL2R common gamma chain 2A is a sequence enabling the co-expression of the two polypeptides, which may be a self cleaving peptide such as a 2A peptide

HuCH1 is a human CH1

IL2bTM is a transmembrane domain from human IL-2R beta

IL2RbENDO is an endodomain from human IL2R beta

IL7RaTM is a transmembrane domain from human IL-7R alpha

IL7RaENDO is an endodomain from human IL-7R alpha

GMCSFRbTM is a transmembrane domain from Human GM-CSFR common beta chain

GMCSFRbEndo is an endodomain from GM-CSFR common beta chain

GMCSFRaTM is a transmembrane domain from Human GF-CSFR alpha

GMCSFRaENDO is an endodomain Derived from Human GM-CSFR alpha

The sequences for the components for making a constitutively active cytokine receptor as shown below as SEQ ID NO. 89 to 101.

(Human Ig Kappa constant domain)
SEQ ID No. 89
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
(Human Hinge)
SEQ ID No. 90
EPKSCDKTHTCPPCP
(Human IgG1 CH1 domain)
SEQ ID No. 91
STKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
(Transmembrane domain from human IL2R common
gamma chain):
SEQ ID No. 92
VVISVGSMGLIISLLCVYFWL
(Transmembrane domain from human IL-2R beta)
SEQ ID No. 93
IPWLGHLLVGLSGAFGFIILVYLLI
(Transmembrane domain from human IL-7R alpha)
SEQ ID No. 94
PILLTISILSFFSVALLVILACVLW
(Transmembrane domain from Human GF-CSFR alpha)
SEQ ID No. 95
NLGSVYIYVLLIVGTLVCGIVLGFLF
(Transmembrane domain from Human GM-CSFR common
beta chain)
SEQ ID No. 96
VLALIVIFLTIAVLLAL
(Endodomain from human IL2R common gamma chain)
SEQ ID No. 97
ERTMPRIPTLKNLEDLVTEYHGNFSAWSGVSKGLAESLQPDYSERLCLVS
EIPPKGGALGEGPGASPCNQHSPYWAPPCYTLKPET
(Endodomain from human IL-2R beta)
SEQ ID No. 98
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV
(Endodomain from human IL-7R alpha)
SEQ ID No. 99
KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ
(Endodomain Derived from Human GM-CSFR alpha)
SEQ ID No. 100
KRFLRIQRLFPPVPQIKDKLNDNHEVEDEIIWEEFTPEEGKGYREEVLTV
KEIT
(Endodomain from GM-CSFR common beta chain)
SEQ ID No. 101
RFCGIYGYRLRRKWEEKIPNPSKSHLFQNGSAELWPPGSMSAFTSGSPPH
QGPWGSRFPELEGVFPVGFGDSEVSPLTIEDPKHVCDPPSGPDTTPAASD
LPTEQPPSPQPGPPAASHTPEKQASSFDFNGPYLGPPHSRSLPDILGQPE
PPQEGGSQKSPPPGSLEYLCLPAGGQVQLVPLAQAMGPGQAVEVERRPSQ
GAAGSPSLESGGGPAPPALGPRVGGQDQKDSPVAIPMSSGDTEDPGVASG
YVSSADLVFTPNSGASSVSLVPSLGLPSDQTPSLCPGLASGPPGAPGPVK
SGFEGYVELPPIEGRSPRSPRNNPVPPEAKSPVLNPGERPADVSPTSPQP
EGLLVLQQVGDYCFLPGLGPGPLSLRSKPSSPGPGPEIKNLDQAFQVKKP
PGQAVPQVPVIQLFKALKQQDYLSLPPWEVNKPGEVC

The constitutively active CCR may comprise a variant of one or more of SEQ ID NO: 89 to 101 having at least 80, 85, 90, 95, 98 or 99% sequence identity, provided that the variant sequence has the required properties. For example, a variant CH or CL sequence should retain the capacity to dimerise with a CL/CH containing-chain. A variant chain from a cytokine receptor endodomain should retain the capacity to trigger cytokine-mediated signalling when coupled with the reciprocal chain for that cytokine receptor.

Nucleic Acid Sequence

The present invention provides a nucleic acid sequence encoding interleukin 12 (IL-12) downstream of a frame-slip motif (FSM) or a translational readthrough motif (TRM).

The nucleic acid sequence may be introduced into a cell such that the cell secretes IL-12 at very low levels.

The nucleic acid sequence may encode a polypeptide comprising a sequence shown as SEQ ID No 1, 24 or 25.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

The present invention provides a nucleic acid construct comprising the nucleic acid sequence of the present invention. The nucleic acid construct may also comprise a nucleic acid sequence encoding a chimeric antigen receptor (CAR) or transgenic TCR.

The nucleic acid construct according may also comprises one or more of the following:
a nucleic acid sequence encoding a dominant negative SHP2;
a nucleic acid sequence encoding a dominant negative TGFβ receptor;
a nucleic acid sequence encoding a chimeric cytokine receptor:
one or more nucleic acid sequence(s) encoding one or more additional cytokine(s) or chemokine(s).

The present invention provides a nucleic acid construct comprising:
(i) a nucleic acid sequence encoding a chimeric antigen receptor (CAR);
(ii) a nucleic acid sequence encoding interleukin 12 (IL-12) downstream of a frame-slip motif (FSM) or a translational readthrough motif (TRM); and
(iii) a nucleic acid sequence encoding IL-15; and/or
(iv) a nucleic acid sequence encoding CXCL12

The present invention provides a nucleic acid construct comprising:
(i) a nucleic acid sequence encoding a chimeric antigen receptor (CAR);
(ii) a nucleic acid sequence encoding interleukin 12 (IL-12) downstream of a frame-slip motif (FSM) or a translational readthrough motif (TRM); and
(iii) a nucleic acid sequence encoding IL-7; and/or
(iv) a nucleic acid sequence encoding CCL19.

The nucleic acid construct may comprise a co-expression sequence between nucleic acid sequences so that the transgenes upstream and downstream of the co-expression sequence are expressed by the cell as separate polypeptides. The co-expression sequence may encode a self-cleaving peptide, which are described in more detail above.

As explained above, a nucleic acid construct for co-expression of a CAR with low-level IL-12 may have the general structure:

CAR-FSM/TRM-coexpr-IL12

in which:

“CAR” is a nucleic acid sequence encoding a chimeric antigen receptor

“FSM/TRM” is a frame-slip motif or a translational readthrough motif

“coexpr” is a sequence enabling the co-expression of the CAR and IL-12 as separate polypeptides; and

“IL-12” is a nucleic acid sequence encoding IL-12 or flexi-IL12

Constructs which comprise a nucleic acid sequence encoding another cytokine or chemokine or a chemokine, may have the general structure:

CAR-FSM/TRM-coexpr1-IL12-coexpr2-CC; or

CAR-FSM/TRM-coexpr1-CC-coexpr2-IL12;

CAR-coexpr1-CC-FSM/TRM-coexpr2-IL12;

CC-coexpr1-CAR-FSM/TRM-coexpr2-IL12

in which:

“CAR” is a nucleic acid sequence encoding a chimeric antigen receptor

“FSM/TRM” is a frame-slip motif or a translational readthrough motif

“coexpr1” and “coexpr2”, which may be the same or different, are sequence enabling the co-expression of the CAR, IL-12 and the cytokine or chemokine as separate polypeptides;

“IL-12” is a nucleic acid sequence encoding IL-12 or flexi-IL12; and

“CC” is a nucleic acid sequence encoding a cytokine (other than IL-12) or a chemokine.

A nucleic acid construct with compound translational readthrough motifs may have the structure:

CAR-FSM/TRM1-coexpr1-CC-FSM/TRM2-coexpr2-IL12

in which:

“CAR” is a nucleic acid sequence encoding a chimeric antigen receptor

“FSM/TRM1” and “FSM/TRM2”, which may be the same of different is/are frame-slip motif(s) or translational readthrough motif(s);

“coexpr1” and “coexpr2”, which may be the same or different, are sequence enabling the co-expression of the CAR, IL-12 and the cytokine or chemokine as separate polypeptides;

“IL-12” is a nucleic acid sequence encoding IL-12 or flexi-IL12; and

“CC” is a nucleic acid sequence encoding a cytokine (other than IL-12) or a chemokine.

Vector

The present invention also provides a vector comprising a nucleic acid sequence or a nucleic acid construct of the invention.

Such a vector may be used to introduce the nucleic acid sequence or construct into a host cell so that it expresses low-level IL-12.

The vector may, for example, be a plasmid or a viral vector, such as a retroviral vector or a lentiviral vector, or a transposon based vector or synthetic mRNA.

The vector may be capable of transfecting or transducing a mammalian cell, for example a T cell or an NK cell.

Kit of Vectors

The invention also provides a kit comprising a plurality of vectors, in which at least one vector comprises a nucleic acid sequence encoding interleukin 12 (IL-12) downstream of a frame-slip motif (FSM) or a translational readthrough motif (TRM).

The vector, or other vectors in the kit may comprise one or more of the following:
a nucleic acid sequence encoding a CAR or transgenic TCR
a nucleic acid sequence encoding a dominant negative SHP2;
a nucleic acid sequence encoding a dominant negative TGFβ receptor;
a nucleic acid sequence encoding a chimeric cytokine receptor:
one or more nucleic acid sequence(s) encoding one or more additional cytokine(s) or chemokine(s).

The kit may comprise:
a first vector which comprises a nucleic acid sequence encoding a CAR and a nucleic acid sequence encoding interleukin 12 (IL-12) downstream of a frame-slip motif (FSM) or a translational readthrough motif (TRM); and
a second vector which comprises a nucleic acid sequence encoding a CAR, a nucleic acid sequence encoding a dominant negative SHP2 and a nucleic acid sequence encoding a dominant negative TGFβ.

Cell

The present invention provides a cell which comprises a nucleic acid sequence encoding interleukin 12 (IL-12) downstream of a frame-slip motif (FSM) or a translational readthrough motif (TRM).

The cell may be a is a tumour infiltrating immune cell, such as a T-cell, natural killer (NK) cell, NKT cell, cytokine-induced killer (CIK) cell, monocyte, macrophage, tumour-infiltrating lymphocyte (TIL), dendritic cell, neutrophil, mast cell, eosinophil or basophil.

The cell may be a cytolytic immune cell such as a T cell and/or or NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+ FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer cells (or NK cells) form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids. They constitute only approximately 0.1% of all peripheral blood T cells.

NKT cells are a subset of T cells that coexpress an αβ T-cell receptor, but also express a variety of molecular markers that are typically associated with NK cells, such as NK1.1. The best-known NKT cells differ from conventional αβ T cells in that their T-cell receptors are far more limited in diversity (‘invariant’ or ‘type 1’ NKT). They and other CD1d-restricted T cells (‘type 2’ NKT) recognize lipids and glycolipids presented by CD1d molecules, a member of the CD1 family of antigen-presenting molecules, rather than peptide-major histocompatibility complexes (MHCs). As such, NKT cells are important in recognizing glycolipids from organisms such as Mycobacterium, which causes tuberculosis.

NKT cells include both NK1.1+ and NK1.1−, as well as CD4+, CD4−, CD8+ and CD8− cells. Natural killer T cells can also share other features with NK cells, such as CD16 and CD56 expression and granzyme production.

Cytokine-induced killer cells (CIK) cells are a group of immune effector cells featuring a mixed T- and natural killer (NK) cell-like phenotype. They are generated by ex vivo incubation of human peripheral blood mononuclear cells (PBMC) or cord blood mononuclear cells with interferon-gamma (IFN-γ), anti-CD3 antibody, recombinant human interleukin (IL-) 1 and recombinant human interleukin (IL)-2.

Typically, immune cells detect major histocompatibility complex (MHC) presented on infected cell surfaces, triggering cytokine release, causing lysis or apoptosis. However, CIK cells have the ability to recognize infected or even malignant cells in the absence of antibodies and MHC, allowing for a fast and unbiased immune reaction. This is of particular importance as harmful cells that are missing MHC markers cannot be tracked and attacked by other immune cells, such as T-lymphocytes. As a special feature, terminally differentiated CD3+CD56+ CIK cells possess the capacity for both MHC-restricted and MHC-unrestricted anti-tumour cytotoxicity. These properties, inter alia, rendered CIK cells attractive as a potential therapy for cancer and viral infections.

A new subclass of NK cells have been created both in vitro and in vivo. These NK cells referred to as cytokine induced memory-like natural killer cells are induced using cytokines, most commonly a mix of IL-12, IL-15, and IL-18. These NK cells are activated by these cytokines to stimulate an infection and induce an adaptive immune response. If cocultured with target cells such as tumour targets, these NK cells have memory-like abilities and are more adapt and effective at mounting a defence.

The cell of the invention may be any of the cell types mentioned above.

The cell may be derived from a blood sample, for example from a leukapheresate.

The cells may be or comprise peripheral blood mononuclear cells (PBMCs).

Cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

The cells may be activated and/or expanded prior to being transduced with nucleic acid encoding the molecules providing the chimeric polypeptide according to the first aspect of the invention, for example by treatment with an anti-CD3 monoclonal antibody.

Pharmaceutical Composition

The cell of the present invention may be administered to a patient as a pharmaceutical composition.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method for Making Cell

In a further aspect, the present invention provides a method for making a cell according to the invention which comprises the step of introducing a nucleic acid sequence, a nucleic acid construct, a vector or a kit of vectors of the invention into a cell.

The nucleic acid sequence, nucleic acid construct, vector or kit of vectors may, for example, be introduced by transduction or transfection in vitro or ex vivo.

The cell may be a cell isolated from a subject, for example a T cell or an NK cell isolated from a subject.

The cell may be autologous or allogeneic.

Method of Treatment

The present invention provides a method for treating a disease which comprises the step of administering a cell composition of the present invention (for example in a pharmaceutical composition as described above) to a subject.

A method for treating a disease relates to the therapeutic use of the cell composition of the present invention. The cell composition may be administered to a subject having an existing disease or condition in order to lessen, reduce or improve at least one symptom associated with the disease and/or to slow down, reduce or block the progression of the disease.

The method for preventing a disease relates to the prophylactic use of the cell composition of the present invention. The cell composition may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing the such cells with a mixture of at least two viral vectors;
(iii) administering the cells from (ii) to a subject.

The present invention also provides a cell composition of the present invention for use in treating and/or preventing a disease.

The invention also relates to the use of a cell composition of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The cells of the composition of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

In particular, the cells of the invention may be used to treat a solid cancer. A solid cancer is a malignancy that forms a discrete tumour mass, for example of brain, breast, prostate, colorectum, kidney; sarcoma; melanoma, in contrast to lymphoproliferative malignancies leukemia, which may diffusely infiltrate a tissue without forming a mass.

The cells of the present invention may be used to treat one of the cancers listed in Table 3.

TABLE 3
Exemplary solid cancer targets and their associated indications

| Target | Indications |
|---|---|
| CD44v6 | Metastatic cancers, colorectal cancers, soft-tissue sarcoma |
| CAIX | Renal cell cancer (RCC), glioblastoma (GBM) |
| CEA | Ovarian cancer, colorectal cancer, cancers of the |
| | gastrointestinal tract, hepatocellular carcinoma (HCC) |
| CD133 | Ovarian cancer, GBM, HCC |
| Claudin-6 | Ovarian cancer |
| Claudin-18.2 | Gastric cancer, pancreatic cancer |
| c-Met | Breast cancer, melanoma, HCC |
| EGFR | Non-small cell lung cancer (NSCLC), GBM, gliomas, |
| | medulloblastoma, soft-tissue sarcoma (STS), osteosarcoma, |
| | Ewing's mesothelioma |
| EGFRvIII | GBM, colorectal cancer, STS, pancreatic cancer |
| EpCAM | HCC, NSCLC, ovarian cancer, breast cancer, gastric cancer, |
| | pancreatic cancer |
| EpA2 | GBM, gliomas |
| Fetal ACho receptor | Osteosarcoma, STS |
| Folate receptor | Ovarian cancer, urothelial cancer |
| GD2 | Neuroblastoma, melanoma, osteosarcoma, STS, Ewing's |
| | sarcoma |
| GPC3 | HCC, SCC |
| GUCY2C | Colorectal cancer |
| HER1 | NSCLC, prostate cancer |
| HER2 | Breast cancer, ovarian cancer, osteosarcoma, STS, GBM, |
| | pediatric CNS cancer, medulloblastoma, gastric cancer, |
| | mesothelioma |
| ICAM-1 | Thyroid |
| IL13Ra2 | GBM, gliomas |
| IL11Ra | Osteosarcoma |
| L1CAM | Ovarian cancer |
| Mesothelin | Pancreatic cancer, ovarian cancer, lung adenocarcinoma |
| | (LUAD), GBM, mesothelioma |
| MUC1 | HCC, NSCLC, pancreatic cancer, breast cancer, glioma, |
| | colorectal, gastric cancer |
| MUC16 | Ovarian cancer |
| NKG2D | Ovarian cancer, osteosarcoma, Ewing's sarcoma |
| NY-ES01 | STS, neuroblastoma, ovarian cancer, breast cancer, GBM, |
| | NSCLC |
| PSA | Prostate cancer, pancreatic cancer |
| PSMA | Prostate cancer, cervical cancer |
| WT1 | Ovarian cancer |

In particular, the cells of the present invention may be used to treat small cell lung cancer (SCLC), melanoma, renal cell cancer (RCC), hepatocellular carcinoma (HCC), ovarian cancer, pancreatic cancer, neuroblastoma, osteosarcoma. In these embodiments, the cells may express a GD2-specific CAR.

Cancer Heterogeneity and Epitope Spreading

The cancer may be heterogeneous. In this respect, the level of expression of the CAR-target antigen may vary between cancer cells. The proportion of cells in the tumour which express the CAR-target antigen may be less than 100%, For example, less than about 95, 90, 80, 70, 60, 50, 40, 30, 20 or 10% of the cells in the tumour may express the target antigen at detectable levels, or at levels recognisable to a CAR-T cell. Some of the cells of the solid cancer may be antigen-negative or antigen-dim, i.e. express low levels of target antigen. An “antigen-dim” target cell may express fewer than about 1000, 750, 500 or 250 copies of the target antigen per cell, on average.

The cells of the present invention are particularly well suited to target heterogenous tumours because of the adjuvant effect of IL-12. IL-12 activates an anti-tumour immune response by activating naïve T cells and macrophages. It has been shown to 1) enhance proliferation of T cells and NK cells, 2) increase cytolytic activities of T cells, NK cells, and macrophages, 3) activate T helper 1 (Th1) cells, and 4) induce production of IFN-γ and other cytokines. IL-12 activates neighbouring CAR-T cells and enhances their cytotoxicity, enhancing their capacity to kill tumour cells with low levels of target antigen

The mechanism of CAR T cell therapy is through direct killing of cells expressing target antigen by T cells expressing the CAR. Without wishing to be bound by theory, the present inventors believe that the simultaneous local expression of IL-12 by CAR-T cells will lead to “epitope spreading” i.e. the priming of immune responses against additional target antigens present on tumour cells. The secretion of IL-12 by CAR-T cells may also induce the infiltration of host immune cells into the tumour mass

IL-12 secreted from T cells thus not only overcomes immune suppression mediated by tumour cells but also provides a conducive environment for epitope spreading, where immune cells diversify to attack multiple tumour-associated targets in addition to the original antigen. The mechanism of action of tumour eradication is through a combination of direct cell killing by CAR-T cells, infiltration of immune cells into the tumour and the induction of an anti-cancer immune response from existing host immune cells against other tumour-associated antigens.

Epitope spreading is important for the treatment of a heterogeneous tumour as it enables the killing of cells which are dim or negative for target antigen. Moreover, it helps prevent immune escape of tumours from CAR-T cell therapy preventing where malignancies may otherwise recur as antigen-negative populations.

The invention will now be further described by way of Examples, which are meant to serve to assist one of ordinary skill in the art in carrying out the invention and are not intended in any way to limit the scope of the invention.

### EXAMPLES

### Example 1—Expression of IL-12 by CAR-T Cells

In a construct encoding two transgenes, the inclusion of a self-cleaving peptide such as an FMDV-derived 2A peptide sequence, leads to approximately equal expression of the two polypeptides. Where the construct co-expresses a CAR and IL-12 this will lead to secretion of high levels of IL-12 by the CAR-T cell (illustrated schematically in FIG. 4A, left-hand diagram).

However, incorporation of a “stop-skip” sequence upstream of the IL-12 encoding sequence should drastically reduce the amount of IL-12 produced by the CAR-T cell (illustrated schematically in FIG. 4A, left-hand diagram).

In order to demonstrate the experimentally, peripheral blood mononuclear cells (PBMCs) were transduced with viruses encoding the following constructs:

CAR-2A-IL-12: comprising a CAR encoding sequence, a 2A self-cleaving peptide, and a sequence encoding flexi-IL-12 (having the sequence shown as SEQ ID No. 1), and

CAR-SS-IL-12: comprising a CAR encoding sequence, a stop-skip sequence (having the sequence shown as SEQ ID No. 37), and a sequence encoding flexi-IL-12 positioned downstream of the stop-skip sequence

Untransduced cells (NT) and PBMCs transduced with either construct, were plated in a 96 well plate at 1×10E⁶ cells/ml and after 48 hours, medium was collected for quantification of released IL-12 by ELISA. The results are shown in FIG. 4B.

Production of IL-12 by the transduced cells was drastically reduced, but still detectable, when the sequence encoding IL-12 was placed downstream of a stop-skip sequence.

### Example 2—Investigating Anti-Tumour Activity of CAR-T Cell Expressing IL-12 In Vivo

Next, the functionality of the SS-IL12 module in vivo was investigated using an immunocompetent mouse model.

CAR-expressing cells were created by transduction of murine PBMCs with vectors expressing the following constructs:

| NAME | CONSTRUCT |
|---|---|
| GD2 CAR | SFGmR.Thy1.1-2A-aGD2_muK666-muCD8STK-muCD28Z |
| CAR-2A-IL-12 | SFGmR.Thy1.1-2A-aGD2_muK666-muCD8STK-muCD28Z-2A- |
| | mfIL12 |
| CAR-SS-IL-12 | SFGmR.Thy1.1-2A-aGD2_muK666-muCD8STK-muCD28Z- |
| | SKIP_TGACAATTA-2A-MUfIL12 |
| EGFRvIII-SS- | SFGmR.THY1.1-2A-aEGFRvIII_MR1-muCD8STK-muCD28Z- |
| IL-12 | SKIP_TGACAATTA-2A-MUfIL12 |

in which “Thy1.1” is a transduction marker; “aGD2_muK666-muCD8STK-muCD28Z” is a second generation murine GD2 CAR; “mfIL-12” is murine flexi-IL-12; “SKIP_TGACAATTA” is a translational readthrough motif placed upstream of the murine flexi-IL-12-encoding sequence; “aEGFRvIII_MR1-muCD8STK-muCD28Z” is a second generation murine CAR against aEGFRvIII, which is used as a negative control.

On the right side of the slide you can see in the chart the drastic weight loss in the CAR-2A-IL12 cohort, due to IL-12 systemic toxicity and these mice had to be euthanized. With regards to tumor growth control; on the right side we can see that tumor growth was not controlled at all in the CAR alone cohort, while mice injected with CAR-SS-IL12 had a drastic reduction an control of the tumor growth, without the toxicity showed by the CAR-2A-IL12 mice cohort.

We can conclude that SS-IL12 module prevents toxicity whilst maintaining potent anti-tumor activity in a challenging murine model

An in vivo assay was used to investigate the anti-tumour activity of T cells expressing a GD2 with or without IL-12 in the murine B16 melanoma model.

B16.F10.GD2 (0.1×10⁶) were administered subcutaneously to mice on day 0. After 7 days 3×10⁶ CAR-T cells were administered intravenously and tumour growth was monitored for the following 14 days.

As shown in FIG. 6, the CAR-2A-IL12 cohort, i.e. mice receiving cells expressing the CAR in combination with IL-12 without a stop-skip sequence, showed drastic weight loss due to IL-12 systemic toxicity. These mice had to be euthanized before day 12. The CAR-SS-IL12 cohort, i.e. mice receiving cells expressing the CAR in combination with ultra-low level IL-12 did not show any significant weight loss or IL-12-related toxicity.

The tumour growth assay results are shown in FIG. 7. Intravenous delivery of CAR T cells expressing a simple GD2 CAR alone had no significant effect on tumour growth (FIG. 7 “CAR”). The introduction of CAR-T cells expressing IL-12, without a translational readthrough motif to control the level of expression, was fatally toxic to the mice (FIG. 7 “CAR-2A”). However, intravenous delivery of CAR T cells co-expressing ultra low-level IL-12, in which the IL-12 encoding sequence was placed downstream of a translational readthrough motif, exhibited potent anti-tumour activity and extended survival of the mice (FIG. 7 “CAR-SS”).

### Example 3—Investigating the Capacity for Ultra-Low IL-12 Expressing CAR-T Cells to Induce Epitope Spreading During an Immune Response

To examine the ability of the ssIL12 module to induce an epitope spreading response in vivo, a target cell line was generated from the murine melanoma cell line B16.F10 transduced to express the two precursor enzymes (GD2 and GD3 synthase) required to synthesise GD2 on the plasmid membrane. This target cell line is 100% positive for GD2 expression and is used to make a heterogenous population of target cells by the addition of non-transduced B16.F10 cells at fixed ratios.

Heterogeneous target populations generated are 10%, 30%, 50%, 70% or 90% GD2-positive and are compared with a homogeneous control of 100% GD2-positive cells. 1E⁵ target cells are subcutaneously injected into an autologous recipient and allowed to engraft for 6 days before the administration of 5 Gray total body irradiation.

The following day, 3E⁶ CAR T cells are injected intravenously. Mice are monitored regularly for signs of toxicity (weight, serum cytokine and general conditioning) and efficacy (calliper measurement of tumour size).

At the time of take down, spleens from the mice are taken for analysis of the proportion of CAR cells, CD4+ T cells, CD8+ T cells, B cells, NK cells, and monocytes. Any remaining tumour at the time of take down is collected for both immunohistochemical analysis (to assess immune infiltrate and tumour morphology) and flow cytometry (to assess the immune infiltrate and proportion and expression levels of GD2 positive tumour).

The degree of epitope spreading is determined by the ability of the module to resolve heterogeneous targets from the recipient mouse and/or the ability of the module to induce immune infiltrate in the tumour mass.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for treating a solid cancer which comprises the step of administering a cell to a subject, wherein the cell comprises a nucleic acid sequence encoding interleukin 12 (IL-12) downstream of a frame-slip motif (FSM) or a translational readthrough motif (TRM).

2. (canceled)

3. A method according to claim 1, wherein the nucleic acid sequence encoding IL-12, encodes “flexi-IL-12”: a fusion between IL-12α and IL-12β subunits, joined by a linker.

4-10. (canceled)

11. A method according to claim 1, wherein the cell is a tumour infiltrating immune cell.

12. (canceled)

13. A method according to claim 1, wherein the cell expresses a chimeric antigen receptor (CAR) or engineered T-cell receptor (TCR)

14. A method according to claim 13, wherein the CAR or engineered TCR binds to one of the following target antigens: disialoganglioside (GD2); epidermal growth factor receptor (EGFR), Epithelial cell adhesion molecule (EpCAM), Glypican 3 (GPC3), human epidermal growth factor receptor (HER2), L1CAM, Mucin 1 (MUC1), Prostate-specific membrane antigen (PSMA).

15-16. (canceled)

17. A method according to claim 1, wherein the cell also expresses a dominant negative SHP2; and/or a dominant negative TGFβ receptor.

18. A method according to claim 1, wherein the cell also expresses a chimeric cytokine receptor.

19. A method according to claim 1, wherein the cell also expresses one or more additional cytokine(s) or chemokine(s).

20. A method according to claim 19, wherein the cell expresses one or more of the following: IL-7, IL-15, CCL19, CXCL12.

21-24. (canceled)

25. A method according to claim 1 for the treatment of small cell lung cancer (SCLC), melanoma, renal cell cancer (RCC), hepatocellular carcinoma (HCC), ovarian cancer, pancreatic cancer, neuroblastoma, osteosarcoma.

26. A method according to claim 1 wherein the cell expresses a CAR or engineered TCR which binds a target antigen and expression of the target antigen on the solid tumour is heterogeneous.

27. A method according to claim 1, wherein the cell induces epitope spreading in an anti-tumour immune response in the subject.

28. A method according to claim 1 for inducing infiltration of immune cells into a tumour mass in the subject, such that they induce an anti-tumour immune response.

29-30. (canceled)

31. A cell which expresses a chimeric antigen receptor (CAR) and comprises;
a nucleic acid sequence encoding interleukin 12 (IL-12) downstream of a frame-slip motif (FSM) or a translational readthrough motif (TRM); and
one or more heterologous nucleic acid sequence(s) encoding one or more additional cytokine(s) or chemokine(s).

32. A cell according to claim 31, wherein the one or more heterologous nucleic acid sequence(s) encode one or more of the following: IL-7, IL-15, CCL19, CXCL12.

33-36. (canceled)

37. A nucleic acid construct comprising:
(i) a nucleic acid sequence encoding a chimeric antigen receptor (CAR);
(ii) a nucleic acid sequence encoding interleukin 12 (IL-12) downstream of a frame-slip motif (FSM) or a translational readthrough motif (TRM); and
(iii) one or more nucleic acid sequence(s) encoding one or more additional cytokine(s) or chemokine(s).

38. A vector comprising a nucleic acid construct according to claim 37.

39. A kit of vectors, comprising:
(i) a vector comprising a nucleic acid sequence encoding a chimeric antigen receptor (CAR);
(ii) a vector comprising a nucleic acid sequence encoding interleukin 12 (IL-12) downstream of a frame-slip motif (FSM) or a translational readthrouqh motif (TRM); and
(iii) vector comprising one or more nucleic acid sequence(s) encoding one or more additional cytokine(s) or chemokine(s).

40. A method for making a cell according to claim 31, which comprises the step of transfecting or transducing a cell with:
i) a nucleic acid sequence encoding a chimeric antigen receptor (CAR);
(ii) a nucleic acid sequence encoding interleukin 12 (IL-12) downstream of a frame-slip motif (FSM) or a translational readthrough motif (TRM); and
(iii) one or more nucleic acid sequence(s) encoding one or more additional cytokine(s) or chemokine(s).


SEQUENCE LISTING (104 sequences)

SEQ ID NO: 1 - protein, 540 aa, Artificial Sequence
  flexi-IL-12 sequence
METDTLLLWVLLLWVPGSTGMWIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPLATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - RNA, 7 nt, Artificial Sequence
  frame-slip motif (FSM) sequence
uuuuuuu

SEQ ID NO: 3 - RNA, 9 nt, Artificial Sequence
  FSM sequence
uuuuuuuga

SEQ ID NO: 4 - RNA, 9 nt, Artificial Sequence
  FSM sequence
uuuuuuuag

SEQ ID NO: 5 - RNA, 9 nt, Artificial Sequence
  FSM sequence
uuuuuuuaa

SEQ ID NO: 6 - RNA, 7 nt, Artificial Sequence
  translational readthrough motif (TRM) sequence
ugacuag

SEQ ID NO: 7 - RNA, 7 nt, Artificial Sequence
  TRM sequence
uagcuag

SEQ ID NO: 8 - RNA, 7 nt, Artificial Sequence
  TRM sequence
uaacuag

SEQ ID NO: 9 - RNA, 9 nt, Artificial Sequence
  TRM sequence
ugacaauua

SEQ ID NO: 10 - RNA, 9 nt, Artificial Sequence
  TRM sequence
uagcaauua

SEQ ID NO: 11 - RNA, 9 nt, Artificial Sequence
  TRM sequence
uaacaauua

SEQ ID NO: 12 - protein, 5 aa, Artificial Sequence
  heavy chain variable region (VH) complementarity determining region (CDR), CDR1
SYNIH

SEQ ID NO: 13 - protein, 16 aa, Artificial Sequence
  VH CDR, CDR2
VIWAGGSTNYNSALMS

SEQ ID NO: 14 - protein, 10 aa, Artificial Sequence
  VH CDR, CDR3
RSDDYSWFAY

SEQ ID NO: 15 - protein, 12 aa, Artificial Sequence
  light chain variable region (VL) CDR, CDR1
RASSSVSSSYLH

SEQ ID NO: 16 - protein, 7 aa, Artificial Sequence
  VL CDR, CDR2
STSNLAS

SEQ ID NO: 17 - protein, 9 aa, Artificial Sequence
  VL CDR, CDR3
QQYSGYPIT

SEQ ID NO: 18 - protein, 5 aa, Artificial Sequence
  VH CDR, CDR1
SSWMN

SEQ ID NO: 19 - protein, 17 aa, Artificial Sequence
  VH CDR, CDR2
RIYPGDGDTNYAQKFQG

SEQ ID NO: 20 - protein, 10 aa, Artificial Sequence
  VH CDR, CDR3
GTGYLWYFDV

SEQ ID NO: 21 - protein, 11 aa, Artificial Sequence
  VL CDR, CDR1
RASQDINENLA

SEQ ID NO: 22 - protein, 7 aa, Artificial Sequence
  VL CDR, CDR2
YTSNRAT

SEQ ID NO: 23 - protein, 9 aa, Artificial Sequence
  VL CDR, CDR3
QQYDNLPFT

SEQ ID NO: 24 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 304 aa, Homo sapiens
WELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPC

SEQ ID NO: 26 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 27 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 29 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 30 - RNA, 10 nt, Artificial Sequence
  multiple stop translational readthrough motif
ugaugacuag

SEQ ID NO: 31 - DNA, 7 bp, Artificial Sequence
  construct DNA sequence encoding a TRM
tgactag

SEQ ID NO: 32 - DNA, 7 bp, Artificial Sequence
  construct DNA sequence encoding a TRM
tagctag

SEQ ID NO: 33 - DNA, 7 bp, Artificial Sequence
  construct DNA sequence encoding a TRM
taactag

SEQ ID NO: 34 - DNA, 9 bp, Artificial Sequence
  construct DNA sequence encoding a TRM
tgacaatta

SEQ ID NO: 35 - DNA, 9 bp, Artificial Sequence
  construct DNA sequence encoding a TRM
tagcaatta

SEQ ID NO: 36 - DNA, 9 bp, Artificial Sequence
  construct DNA sequence encoding a TRM
taacaatta

SEQ ID NO: 37 - DNA, 9 bp, Artificial Sequence
  construct DNA sequence encoding a TRM
tgactagca

SEQ ID NO: 38 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 39 - protein, 7 aa, Artificial Sequence
  consensus Tobacco Etch Virus (TEV) cleavage site
ENLYFQS

SEQ ID NO: 40 - protein, 8 aa, Artificial Sequence
  motif involved in cleavage activity
  misc_feature (2)..(2): Xaa can be any naturally occurring amino acid
  misc_feature (4)..(4): Xaa can be any naturally occurring amino acid
DXEXNPGP

SEQ ID NO: 41 - protein, 8 aa, Artificial Sequence
  cleavage site motif
DIETNPGP

SEQ ID NO: 42 - protein, 8 aa, Artificial Sequence
  cleavage site motif
DVETNPGP

SEQ ID NO: 43 - protein, 8 aa, Artificial Sequence
  cleavage site motif
DVEMNPGP

SEQ ID NO: 44 - protein, 8 aa, Artificial Sequence
  cleavage site motif
DVESNPGP

SEQ ID NO: 45 - protein, 8 aa, Artificial Sequence
  cleavage site motif
DMESNPGP

SEQ ID NO: 46 - protein, 8 aa, Artificial Sequence
  cleavage site motif
DVEENPGP

SEQ ID NO: 47 - protein, 8 aa, Artificial Sequence
  cleavage site motif
DIELNPGP

SEQ ID NO: 48 - protein, 8 aa, Artificial Sequence
  cleavage site motif
DIEQNPGP

SEQ ID NO: 49 - protein, 8 aa, Artificial Sequence
  cleavage site motif
DSEFNPGP

SEQ ID NO: 50 - protein, 19 aa, Artificial Sequence
  cleavage sequence
LLNFDLLKLAGDVESNPGP

SEQ ID NO: 51 - protein, 19 aa, Artificial Sequence
  cleavage sequence
LLNFDLLKLAGDVQSNPGP

SEQ ID NO: 52 - protein, 19 aa, Artificial Sequence
  cleavage sequence
LLNFDLLKLAGDVEINPGP

SEQ ID NO: 53 - protein, 19 aa, Artificial Sequence
  cleavage sequence
LLNFDLLKLAGDVEFNPGP

SEQ ID NO: 54 - protein, 19 aa, Artificial Sequence
  cleavage sequence
LLNFDLLKLAGDVESHPGP

SEQ ID NO: 55 - protein, 19 aa, Artificial Sequence
  cleavage sequence
LLNFDLLKLAGDVESEPGP

SEQ ID NO: 56 - protein, 19 aa, Artificial Sequence
  cleavage sequence
LLNFDLLKLAGDVESQPGP

SEQ ID NO: 57 - protein, 19 aa, Artificial Sequence
  cleavage sequence
LLNFDLLKLAGDVESNPGG

SEQ ID NO: 58 - protein, 20 aa, Artificial Sequence
  cleavage site, 2A-like sequence
YHADYYKQRLIHDVEMNPGP

SEQ ID NO: 59 - protein, 20 aa, Artificial Sequence
  cleavage site, 2A-like sequence
HYAGYFADLLIHDIETNPGP

SEQ ID NO: 60 - protein, 20 aa, Artificial Sequence
  cleavage site, 2A-like sequence
QCTNYALLKLAGDVESNPGP

SEQ ID NO: 61 - protein, 19 aa, Artificial Sequence
  cleavage site, 2A-like sequence
ATNFSLLKQAGDVEENPGP

SEQ ID NO: 62 - protein, 19 aa, Artificial Sequence
  cleavage site, 2A-like sequence
AARQMLLLLSGDVETNPGP

SEQ ID NO: 63 - protein, 20 aa, Artificial Sequence
  cleavage site, 2A-like sequence
RAEGRGSLLTCGDVEENPGP

SEQ ID NO: 64 - protein, 20 aa, Artificial Sequence
  cleavage site, 2A-like sequence
TRAEIEDELIRAGIESNPGP

SEQ ID NO: 65 - protein, 20 aa, Artificial Sequence
  cleavage site, 2A-like sequence
TRAEIEDELIRADIESNPGP

SEQ ID NO: 66 - protein, 20 aa, Artificial Sequence
  cleavage site, 2A-like sequence
AKFQIDKILISGDVELNPGP

SEQ ID NO: 67 - protein, 20 aa, Artificial Sequence
  cleavage site, 2A-like sequence
SSIIRTKMLVSGDVEENPGP

SEQ ID NO: 68 - protein, 20 aa, Artificial Sequence
  cleavage site, 2A-like sequence
CDAQRQKLLLSGDIEQNPGP

SEQ ID NO: 69 - protein, 20 aa, Artificial Sequence
  cleavage site, 2A-like sequence
YPIDFGGFLVKADSEFNPGP

SEQ ID NO: 70 - protein, 18 aa, Artificial Sequence
  2A peptide from Thosea asigna virus capsid protein
EGRGSLLTCGDVEENPGP

SEQ ID NO: 71 - protein, 57 aa, Artificial Sequence
  cleavage sequence
VTELLYRMKRAETYCPRPLAIHPTEARHKQKIVAPVKQTLNFDLLKLAGDVESNPGP

SEQ ID NO: 72 - protein, 40 aa, Artificial Sequence
  cleavage sequence
LLAIHPTEARHKQKIVAPVKQTLNFDLLKLAGDVESNPGP

SEQ ID NO: 73 - protein, 33 aa, Artificial Sequence
  cleavage sequence
EARHKQKIVAPVKQTLNFDLLKLAGDVESNPGP

SEQ ID NO: 74 - protein, 24 aa, Artificial Sequence
  cleavage sequence
APVKQTLNFDLLKLAGDVESNPGP

SEQ ID NO: 75 - protein, 118 aa, Artificial Sequence
  Humanised KM666 VH sequence
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSS

SEQ ID NO: 76 - protein, 108 aa, Artificial Sequence
  Humanised KM666 VL sequence
ENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIK

SEQ ID NO: 77 - protein, 119 aa, Artificial Sequence
  Humanised 7A12 VH sequence
QVQLVQSGAEVKKPGASVKVSCKASGYTFTSSWMNWVRQAPGQGLEWMGRIYPGDGDTNYAQKFQGRVTMTRDTSISTAYMELSRLRSDDTAVYYCARGTGYLWYFDVWGQGTMVTVSS

SEQ ID NO: 78 - protein, 108 aa, Artificial Sequence
  Humanised 7A12 VL sequence
EIVLTQSPATLSLSPGERATLSCRASQDINENLAWYQQKPGQAPRLLIYYTSNRATGIPARFSGSGSGRDFTLTISSLEPEDFAVYYCQQYDNLPFTFGQGTKVEIKR

SEQ ID NO: 79 - protein, 41 aa, Artificial Sequence
  CD28 endodomain
RSKRSRLLHSDYMNMTPRRPGPTRKHYQPYAPPRDFAAYRS

SEQ ID NO: 80 - protein, 112 aa, Artificial Sequence
  CD3z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 82 - protein, 214 aa, Artificial Sequence
  SHP-1 SH2 complete domain
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYY

SEQ ID NO: 83 - protein, 97 aa, Artificial Sequence
  SHP-1 SH2 domain 1
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 84 - protein, 104 aa, Artificial Sequence
  SHP-1 SH2 domain 2
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 85 - protein, 97 aa, Artificial Sequence
  SHP-2 first SH2 domain
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 86 - protein, 105 aa, Artificial Sequence
  SHP-2 second SH2 domain
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 87 - protein, 211 aa, Artificial Sequence
  SHP-2 tandem SH2 domains
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 88 - protein, 177 aa, Artificial Sequence
  dominant negative TGFbeta receptor II
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 89 - protein, 107 aa, Artificial Sequence [sequence omitted]

SEQ ID NO: 90 - protein, 15 aa, Artificial Sequence
  Human Hinge
EPKSCDKTHTCPPCP

SEQ ID NO: 91 - protein, 97 aa, Artificial Sequence
  Human IgG1 CH1 domain
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 92 - protein, 21 aa, Artificial Sequence
  Transmembrane domain from human IL2R common gamma chain
VVISVGSMGLIISLLCVYFWL

SEQ ID NO: 93 - protein, 25 aa, Artificial Sequence
  Transmembrane domain from human IL-2R beta
IPWLGHLLVGLSGAFGFIILVYLLI

SEQ ID NO: 94 - protein, 25 aa, Artificial Sequence
  Transmembrane domain from human IL-7R alpha
PILLTISILSFFSVALLVILACVLW

SEQ ID NO: 95 - protein, 26 aa, Artificial Sequence
  Transmembrane domain from Human GF-CSFR alpha
NLGSVYIYVLLIVGTLVCGIVLGFLF

SEQ ID NO: 96 - protein, 17 aa, Artificial Sequence
  Transmembrane domain from Human GM-CSFR common beta chain
VLALIVIFLTIAVLLAL

SEQ ID NO: 97 - protein, 86 aa, Artificial Sequence
  Endodomain from human IL2R common gamma chain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 98 - protein, 286 aa, Artificial Sequence
  Endodomain from human IL-2R beta
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 99 - protein, 195 aa, Artificial Sequence
  Endodomain from human IL-7R alpha
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 100 - protein, 54 aa, Artificial Sequence
  Endodomain Derived from Human GM-CSFR alpha
KRFLRIQRLFPPVPQIKDKLNDNHEVEDEIIWEEFTPEEGKGYREEVLTVKEIT

SEQ ID NO: 101 - protein, 437 aa, Artificial Sequence
  Endodomain from GM-CSFR common beta chain
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 102 - protein, 6 aa, Artificial Sequence
  CXCL12 loop region motif
RFFESH

SEQ ID NO: 103 - protein, 8 aa, Artificial Sequence
  cleavage site motif
DVELNPGP

SEQ ID NO: 104 - protein, 4 aa, Artificial Sequence
  Mucin 1 motif
PDTR